(19) United States
(12) Patent Application Publication
(10) Pub. No.: US 2012/0022380 A1
(43) Pub. Date: Jan. 26, 2012

(54) METHODS AND APPARATUS FOR ASSESMENT AND TREATMENT OF BODY CAVITIES

(76) Inventor: Ary S. CHERNOMORSKY, Walnut Creek, CA (US)

(21) Appl. No.: 13/009,843

(22) Filed: Jan. 19, 2011

Related U.S. Application Data
(60) Provisional application No. 61/296,122, filed on Jan. 19, 2010.

(51) Int. Cl. A61B 6/00

(52) U.S. Cl. 600/476

(57) ABSTRACT

Methods and apparatus for detection, assessment and optionally treatment of the cancerous tissue in the natural and manmade body cavities. An expandable cavity assessment device that is coupled with cancer detecting elements is placed in a cavity in close proximity to the site of cancerous tissue. The cavity assessment device may receive an optical fiber, or other type of energy conduit and potentially a radioactive source for interstitial radiation therapy. The cavity assessment device may be equipped with a balloon member coupled with electro conductive elements. Further, a system facilitating substance communication with the internal surface of the cavity is provided.

### CROSS-REFERENCE TO RELATED APPLICATIONS

The present application claims priority to copending and commonly assigned U.S. provisional application Ser. No. 61/296,122 filed on Jan. 19, 2010, which is incorporated herewith by reference.

### BACKGROUND OF THE INVENTION

### Field of the Invention

The field of the present inventions encompasses medical devices and methods for the analysis, assessment and treatment of natural and manmade body cavities and lumens. Embodiments of the present inventions are also directed to the detection and treatment of the cancerous tissues in natural and manmade body cavities and lumens.

### SUMMARY OF THE INVENTION

According to an embodiment thereof, the present invention is a method of characterizing tissue. The method may include steps of providing a catheter defining a proximal portion and a distal portion, the distal portion including a carrier that is configured to deliver an agent that is sensitive to abnormal cells; inserting the catheter in a cavity within the tissue; causing the carrier to bring the abnormal cell sensitive agent into contact with a surface of the cavity, and illuminating the surface of the cavity.

According to further embodiments, the method may further include rotating the distal portion of the catheter within the cavity and causing the carrier to bring the abnormal cell sensitive agent into contact with another region of the surface of the cavity prior to the illuminating step. A step of characterizing the tissue of the interior surface of the cavity depending upon a characteristic of a light reflected from within the cavity or absorbed by the surface of the cavity may also be carried out. The characterizing step may include determining whether the surface of the cavity includes cancer or other abnormal cells, depending upon characteristics of the light reflected from within or absorbed by the cavity. A step may be carried out to bring the distal portion of the catheter into close conformance with a surface of the cavity using a source of vacuum. The providing step may be carried out with the abnormal cell sensitive agent including a dye, such as a pH-sensitive dye, for example. Alternatively, the providing step may be carried out with the abnormal cell sensitive agent including a fluorescent dye or a fluorescent dye and a selected antibody having an affinity to abnormal cells.

The providing step may be carried out with the catheter including a plurality of carriers that include pH-sensitive elements. The providing step may also be carried out with the distal portion of the catheter including a light source. The illuminating step may be carried out by inserting a scope within the catheter to the cavity, the scope including a light source.

The method may also, according to further embodiments of the present inventions, further include a step of displaying an image produced from light reflected from within or absorbed by the cavity on a display coupled to the scope. A gradient of pH across the surface of the cavity may be determined, with the gradient being indicative of a presence and distribution of cancerous or other abnormal cells within the cavity.

The providing step may be carried out with the carrier including a plurality of needle-like structures configured to deliver the abnormal cell sensitive agent to the cavity. The providing step may be carried out with the distal portion including a lumen in fluid communication with a reservoir of abnormal cell sensitive agent and the plurality of needle-like structures. The providing step may also be carried out with the carrier including one or more balloons configured to deliver the abnormal cell sensitive agent to a surface of the cavity. The balloon(s) may be configured to be selectively expandable and collapsible. The balloon(s) may be configured to deliver the abnormal cell sensitive agent over a surface of the cavity that is substantially coextensive with an external surface of the balloon(s). The providing step may be carried out with a surface of the balloon(s) including surfaces that define a plurality of openings, the plurality of openings being coupled to a source of vacuum. The providing step may be carried out with the distal portion of the catheter including a cavity expander having a preset geometry, the cavity expander including surfaces that define a plurality of openings that are configured to couple to a source of vacuum. The providing step may also be carried out with the providing step with the distal portion including a plurality of selectively expandable and collapsible balloons. The size and/or shape of cavity expander may be configured to substantially match the size and/or shape of the cavity, but for an opening (such as a notch, for example) that is configured to enable the carrier to selectively collapse therein and emerge therefrom to bring the abnormal cell sensitive agent into contact with the surface of the cavity.

According to another embodiment thereof, the present invention is a catheter that includes a proximal portion and a distal portion; a carrier, the carrier being configured to deliver an agent that is sensitive to abnormal cells to a surface of a cavity within biological tissue; a source of illumination configured to illuminate abnormal cell sensitive agent delivered to the surface of the cavity, and an interface configured to couple to a display, the interface being configured to enable the display to display an image representative to light reflected from or absorbed by the abnormal cell sensitive agent delivered to the surface of the cavity.

The catheter (e.g., tissue assessment device), according to further embodiments, may further include a plurality of carriers configured to deliver abnormal cell sensitive agent to a surface of the cavity. The source of illumination may be coupled to the distal portion of the catheter. At least the distal portion may include a surface configured to define an interior lumen through which a scope including a light source is insertable. The carrier may include a plurality of needle-like structures configured to deliver the abnormal cell sensitive agent to at least the surface of the cavity. At least the distal portion may include a surface that defines a lumen in fluid communication with a reservoir of abnormal cell sensitive agent and the plurality of needle-like structures. The carrier may be coupled with (contain or otherwise configured to deliver) an abnormal cell sensitive agent. For example, the abnormal cell sensitive agent may include a dye, such as a pH-sensitive dye, a photosensitive dye such as a fluorescent dye or a fluorescent dye and a selected antibody having an affinity to abnormal cells.

The catheter may further include a balloon and the carrier may be constituted of or include the outer surface of the balloon, and at least a portion of the outer surface of the balloon may be configured to deliver the abnormal cell sensitive agent to at least the surface of the cavity. The balloon may be configured to be selectively expandable and collapsible. The balloon may include surfaces that define a plurality of openings, and these plurality of openings may be configured to couple to a source of vacuum. The catheter may further include a cavity expander having a preset geometry. A plurality of selectively expandable and collapsible balloons may be disposed on the cavity expander. The size and/or shape of cavity expander may be configured to substantially match the size and/or shape of the cavity, but for an opening configured to enable the carrier to expand and emerge therefrom to bring the abnormal cell sensitive agent into contact with a surface of the cavity facing the carrier. The cavity expander may defines a plurality of radially oriented openings, each being configured to enable a respective carrier to expand and emerge therefrom and come into contact with a surface of the cavity. The carrier may include a porous matrix that may be coupled to, loaded with or otherwise configured to deliver abnormal cell sensitive agent. For example, the carrier may include one or more expandable and collapsible loops and the proximal portion of the catheter may include an actuator coupled to the carrier. A plurality of balloons may be coupled to the distal portion, and the distal portion may include a plurality of carriers, the plurality of balloons each being configured to assume an expanded state in which each of the plurality of carriers is nestled between at least two (e.g., adjacent ones) of the expanded balloons and a collapsed state in which the plurality of carriers are configured to come into contact with a surface of the cavity.

According to still another embodiment thereof, the present invention is a method of characterizing tissue. The method may include steps of providing a catheter defining a proximal portion and a distal portion, the distal portion including a surface that is configured to contact a surface of a cavity within the tissue, the distal portion being configured to selectively elicit a physiological response from tissue within the cavity; inserting the catheter in the cavity within the tissue; causing the physiological response to occur, and observing the physiological response and characterizing the tissue depending upon the observed physiological response.

According to further embodiments, the providing step may be carried out with the distal portion being configured to substantially conform to a shape and/or size of the cavity. The providing step may be carried out with at least a portion of the distal portion being expandable and collapsible. The providing step may be carried out with at least a portion of the distal portion having surfaces that define a plurality of vacuum orifices that are configured to couple to a source of vacuum, and the distal portion may be configured to drawn the surface of the cavity toward the distal portion when vacuum is applied. The providing step may be carried out with the distal portion including a porous matrix. The providing step may be carried out with the distal portion being configured to selectively elicit the physiological response using a dye, such as, for example, a pH-sensitive dye, a photosensitive dye such as a fluorescent dye or a fluorescent dye with a selected antibody having an affinity to abnormal cells.

The providing step may be carried out with the distal portion being configured to deliver a reagent that is sensitive to malignant cells. The providing step may be carried out with the distal portion being configured to deliver a reagent having sensitivity to an ionic strength of the tissue within the cavity. Alternatively, the providing step may be carried out with the distal portion being configured to deliver a reagent having sensitivity to conductivity of the tissue within the cavity. The providing step may be carried out with the distal portion being configured to deliver a reagent configured to cause light to reflect or be absorbed differently from or by the tissue within the cavity, depending upon a characteristic of the tissue. The surface of the distal portion may include at least one needle-like structure configured to deliver at least one of a reagent and a therapeutic agent to tissue within the cavity. The surface of the distal portion may include at least two electrodes and the physiological response may be electrical in nature. The physiological response may be one of conductivity and impedance. The providing step may be carried out with the distal portion being configured to deliver an iontophoretic agent to the cavity.

Still another embodiment of the present invention is a method of mapping a post-surgical cavity that may include steps of providing a catheter, the catheter having a distal portion configured to substantially occupy a volume of space delimited by a surface of the cavity, the distal portion being at least partially translucent to a predetermined source of light, the distal portion being further configured to deliver a reagent from a surface of the distal portion to the surface of the cavity; inserting the catheter within the cavity and delivering the reagent to at least a surface of the cavity, and illuminating the cavity with the predetermined source of light through the at least partially translucent distal portion.

The method may also include a step of displaying an image of a light reflected from within the cavity on a display that is external and coupled to the catheter. The providing step may be carried out with the distal portion including a porous matrix. The providing step may be carried out with the distal portion in fluid communication with a reservoir of the reagent. The providing step may be carried out with the distal portion being expandable such that it substantially occupies the volume of space delimited by the cavity and collapsible such that it then occupies a smaller volume than the volume delimited by the cavity. The providing step may be carried out with the distal portion including surfaces that define vacuum orifices that are configured to couple to a source of vacuum, and the inserting step may include a step of adhering the surface of the cavity to the distal portion using vacuum. The delivering step may be configured to deliver the reagent to most (or a substantial portion of) of the entire surface of the cavity at the same time. The delivering step may deliver the reagent across at least one band on a surface of the cavity, and the delivering step further may include at least one step of rotating the distal portion within the cavity and re-delivering the reagent across at least one other band on a surface of the cavity. The providing step may be carried out with the distal portion including a user-actuated collapsible and expandable loop, and with the distal portion defining a preset geometry that substantially conforms to a size and/or shape of the cavity, the distal portion further defining at least one opening (such as a radial notch, for example) within which the loop may be configured to collapse and expand.

According to yet another embodiment, the present invention is a catheter. The catheter may include a proximal portion, and a distal portion coupled to the proximal portion and configured to be inserted within a cavity within biological tissue, the distal portion including a portion that may be at least partially transparent to a predetermined light and that may be configured to deliver a reagent to the cavity, the distal portion being further configured to include and/or receive a source of the predetermined light to illuminate a surface of the cavity to which the reagent has been delivered through the at least partially transparent portion.

The catheter may further include an interface, the interface being configured to couple to a display to enable light reflected from within the cavity to be represented on the display. The at least partially transparent portion may include, be or provide support for a three dimensional porous matrix. The at least partially transparent portion may be preloaded with the reagent. The at least partially transparent portion may be in fluid communication with a reservoir of the reagent. The distal portion may be expandable such that it substantially occupies the volume of space delimited by the cavity and may be collapsible such that it then occupies a smaller volume than the volume delimited by the cavity. The distal portion may include surfaces that define vacuum orifices that are configured to couple to a source of vacuum. The at least partially transparent portion may be configured to deliver the reagent to most of the entire surface of the cavity at the same time. The catheter may further include a first electrode coupled to the distal portion that is configured to be electrically charged to a first polarity and a second electrode that is configured to couple to the tissue and that is configured to be charged to a second polarity. The reagent may be driven into the tissue when the first and second electrodes are charged and establish a potential difference therebetween.

Yet another embodiment of the present inventions is a method that may include steps of providing a catheter having a proximal and a distal portion, the distal portion including an expandable and collapsible balloon whose outer surface is coupled to a reagent that is sensitive to abnormal cells; inserting the distal portion of the catheter in a cavity within biological tissue with the distal portion in a collapsed state; expanding the balloon so that the outer surface of the balloon comes into intimate contact with a surface of the cavity; collapsing the balloon and retracting the catheter from the cavity, and expanding the balloon and treating the cavity using the reagent-coated surface of the expanded balloon as a map to corresponding locations of remaining abnormal cells within the cavity.

According to further embodiments, the providing step may be carried out with the reagent including a dye, such as a pH-sensitive dye, a photosensitive dye such as, for example, a fluorescent dye or a fluorescent dye and a selected antibody having an affinity to abnormal cells. The treating step may include resecting further tissue within the cavity at locations indicated by a corresponding location on the surface of the balloon where the reagent reacted to the presence abnormal cells within the cavity. The treating step may include, for example, delivering a therapeutic agent to the cavity. The therapeutic agent may include, for example, a source of radiation and/or a chemotherapy agent. The providing step may be carried out with the distal portion including a surface that defines a central lumen and may include a plurality of needle-like structures on a surface of the balloon. The treating step may include re-inserting the distal portion of the catheter in the cavity and delivering the therapeutic agent to the cavity through the central lumen and/or at least one of the plurality of needle-like structures.

### BRIEF DESCRIPTION OF THE DRAWINGS

FIG. 1A is a representation of a female breast, shown in such a manner that a tumor therein is visible. FIG. 1A also shows a tumor removed from the breast as a result of a lumpectomy procedure and the post-surgical cavity left behind as a result of the lumpectomy procedure.

FIG. 1B shows a tissue assessment device according to embodiments of the present inventions, inserted within the post-surgical cavity;

FIG. 1C shows further aspects of a tissue assessment system, according to embodiments of the present inventions;

FIG. 1D is a block diagram illustrating aspects of the embodiments of the present invention;

FIG. 2A shows a bio-photometric cavity assessment device and system, according to a further embodiment of the present inventions.

FIG. 2B shows the bio-photometric cavity assessment device of FIG. 2A, inserted within a tissue cavity or lumen, according to further aspects of embodiments of the present invention;

FIG. 2C is a representation of a cavity assessment device and system for tumor detection, according to further embodiments of the present inventions;

FIG. 2D provides two exemplary shapes for an abnormal cell sensitive agent carrier for cavity assessment devices and systems according to further embodiments of the present invention.

FIG. 3A shows a balloon-based cavity assessment device, according to embodiments of the present inventions, that is to be inserted through a manmade channel through breast tissue, into a post-surgical cavity.

FIG. 3B shows the balloon-based cavity assessment device of FIG. 3A, inserted into the post-surgical cavity.

FIG. 3C illustrates the balloon-based cavity assessment device of FIGS. 3A and 3B in operation, utilizing the three-dimensional balloon mapping technique according to embodiments of the present invention.

FIG. 3D shows a cavity assessment device and system utilizing a balloon mapping technique based on visual changes in color for respective areas of the balloon surface, using computerized image analysis and reconstruction, according to further embodiments of the present invention;

FIG. 4A shows an exemplary distal portion of the cavity assessment device according to another embodiment of the present inventions;

FIG. 4B is another view of the distal portion of the cavity assessment device of FIG. 4A, in which the carrier or loop is in its expanded state;

FIG. 4C is another view of the distal portion of the cavity assessment device of FIG. 4A, in which the carrier or loop is in its retracted state within the distal portion;

FIG. 4D shows another embodiment of the distal portion of the cavity assessment device according to embodiments of the present invention, featuring a multi-loop arrangement with a preset geometry cavity expander and vacuum assistance;

FIG. 4E is a front view of the distal portion of the cavity assessment device of FIG. 4D;

FIG. 4F shows another embodiment of the distal portion of the cavity assessment device according to embodiments of the present invention, featuring a single loop device coupled with a fixed geometry cavity expander, in which the carrier or loop is equipped with a plurality of needle-like structures;

FIG. 4G is a view of the distal portion of the cavity assessment device of FIG. 4F, showing the carrier in cross-section in an expanded configuration and coupled with a needle-like structure;

FIG. 4H shows the single loop of the cavity assessment device shown in FIG. 4F, detailing the plurality needle-like structures that are part of, disposed on or coupled to the single carrier or loop, according to embodiments of the present invention;

FIG. 4I is a cross-sectional view of the distal portion of the cavity assessment device of FIG. 4F, showing the carrier in a contracted, collapsed or non-extended configuration;

FIG. 4J shows aspects of another embodiment of the cavity assessment device according to the present inventions, including a multi-loop device coupled with a fixed geometry, a cavity expander where one or more of the carriers of the multi-loop device is equipped with plurality of needle-like structures;

FIG. 5A shows a cross-sectional view of the distal portion of a cavity assessment device inserted in a post-surgical cavity within a breast, to illustrate further aspects of embodiments of the present cavity assessment devices and steps of the present cavity assessment methods, according to further embodiments of the present inventions;

FIG. 5B is a cross-sectional view of the distal portion of a cavity assessment device of FIG. 5A, shown in a configuration in which the carrier or loop is in an expanded state and aligned with a target tissue (such as, e.g., cancerous tissue or other tumor), to enable the needle-like structures thereof to inject reagents and/or other therapeutic agents therein;

FIG. 5C shows the cross-sectional view of the distal portion of a cavity assessment device of FIG. 5A, shown in rotation and in a configuration in which a carrier or loop thereof is in a contracted configuration;

FIG. 5D shows the cross-sectional view of the distal portion of a cavity assessment device of FIG. 5C, in a configuration in which the distal portion of the cavity assessment device has been rotated such that the carrier or loop is aligned with a second target tissue, to enable the needle-like structure(s) thereof to inject a reagent and/or other therapeutic agent(s) into the second target tissue;

FIG. 6A is a side view of a distal portion of a cavity assessment device featuring a plurality of carriers/loops and a variable cavity expander that includes a fixed geometry portion and a plurality of expandable and collapsible balloons, according to further embodiments of the present inventions;

FIG. 6B is a front view of the distal portion of a cavity assessment device of FIG. 6A, in which the plurality of balloons are in their expanded state;

FIG. 6C shows the front view of the distal portion of a cavity assessment device of FIG. 6B, in a configuration in which the plurality of balloons are their collapsed configuration;

FIG. 6D shows a cross-sectional frontal view of a cavity assessment device showing a carrier thereof having one or more needle-like structures formed as part of or coupled thereto, according to embodiments of the present invention;

FIG. 6E shows a cross-sectional frontal view of a cavity assessment device having a fixed geometry portion, a single carrier and a plurality of expandable and collapsible balloons, according to embodiments of the present invention;

FIG. 7A illustrates use of a cavity assessment system utilizing selective fluorescent agent uptake, according to further embodiments of the present invention;

FIG. 7B is a cross-sectional view of the cavity assessment system shown in FIG. 7A, in a configuration in which a plunger of a fluorescent agent-containing reservoir is in a proximal position;

FIG. 7C is a cross-sectional view of the cavity assessment system of FIGS. 7A and 7B, in a configuration in which the plunger of the fluorescent agent-containing reservoir has been pushed to a distal position to deliver the fluorescent agent to the three-dimensional surface of the distal portion of the cavity assessment device, according to embodiments of the present inventions;

FIG. 8A is a schematic illustrating a cavity assessment system utilizing electrophysiological properties such as iontophoretic agent uptake, according to further embodiments of the present inventions;

FIG. 8B is a cross-sectional view of the cavity assessment system shown in FIG. 8A, in a configuration in which a plunger of a iontophoretic agent-containing reservoir is in a proximal position;

FIG. 8C is a cross-sectional view of the cavity assessment system of FIGS. 8A and 8B, in a configuration in which the plunger of the iontophoretic agent-containing reservoir has been pushed to a distal position to deliver the iontophoretic agent to the electro-conductive three-dimensional surface of the distal portion of the cavity assessment device, according to further embodiments of the present inventions;

FIG. 9A is a schematic illustrating a cavity assessment system utilizing electrophysiological properties such impedance and/or conductivity measurements, according to further embodiments of the present invention.

FIG. 9B includes cross sectional views of the cavity assessment device of FIG. 9A, showing a distal portion thereof having an electro conductive three-dimensional actuating surface including a plurality of needle electrodes, according to further embodiments of the present inventions;

FIG. 9C is a cross section view of the cavity assessment device of FIGS. 9A and 9B, with the distal portion thereof shown in its expanded configuration, according to embodiments of the present invention;

FIG. 10A shows a cavity treatment device of the present cavity treatment system in use within a post-surgical cavity in its expanded configuration, according to embodiments of the present inventions;

FIG. 10B shows a cavity treatment system according to further embodiments of the present inventions, including an electro-conductive three-dimensional actuating surface, a therapeutic agent reservoir loaded with a therapeutic agent, a source of electric current and external electrodes.

### DETAILED DESCRIPTION

In the following detailed description of exemplary embodiments of the invention, reference is made to the accompanying drawings, which form a part hereof, and in which is shown by way of illustration specific exemplary embodiments in which the invention may be practiced. These embodiments are described in sufficient detail to enable those skilled in the art to practice the invention, and it is to be understood that other embodiments may be utilized and that logical, mechanical, electrical and other changes may be made without departing from the spirit or scope of the present invention. The following detailed description is, therefore, not to be taken in a limiting sense, and the scope of the present invention is defined only by the appended claims.

Many medical procedures require the surgical formation and maintenance of a cavity within a patient's body. For example, the treatment of certain tumors may require a multi-faceted approach that includes a combination of surgery, radiation therapy and chemotherapy. In such an approach, after an initial surgical procedure has been performed to remove as much of a tumor as possible, radiation and chemotherapy are often performed to kill remaining cancerous cells that could not be removed surgically. The amount and distribution of these remaining cancerous cells within the post-surgical cavity or lumen is conventionally assessed by sending the dissected and excised tissue sample out to a pathology lab for histo-pathological analysis. The process of sending out the excised sample to a lab (even one located on-premises), and waiting for the returned analysis is, however, time consuming. Intraoperative or immediate postoperative assessment of the surgical cavity is, therefore, highly desirable to enable the operating physicians to determine whether immediate or subsequent treatment is necessary. In this context, an immediate treatment may include the surgical removal of detected remaining cancerous tissues and subsequent treatment may include providing therapeutic materials directly into or adjacent to surgery site, in direct or close contact with the targeted tissues.

In the case of radiation therapy, one of the more effective treatment methods is brachytherapy in which a source of radiation energy is placed within the body of the patient at the site of the removed tumor to substantially evenly treat the region that formerly surrounded the surgically removed tumor. In addition to or instead of radiation therapy, therapeutic chemical compounds may be used to kill cancerous cells located in the vicinity of a surgically removed tumor.

Embodiments of the present inventions provide methods and devices for characterizing tissue surrounding a surgical resection cavity (herein also referred to interchangeably herein as a post-surgical cavity, cavity or lumen) in solid tissue, such as a lumpectomy cavity formed in a breast following breast cancer surgery or a natural cavity such as rectum, and subsequent additional treatment. The assessment may comprise detection of the cancerous tissue, employing chemical, electrochemical, electrophysiological and optical methods or combinations of such, and may be used as an aid in planning an immediate or subsequent treatment of the surrounding tissue. In addition, the methods and apparatus may further provide for treatment of the surrounding tissue, using such modalities as local radiation therapy (brachytherapy) or local chemotherapy.

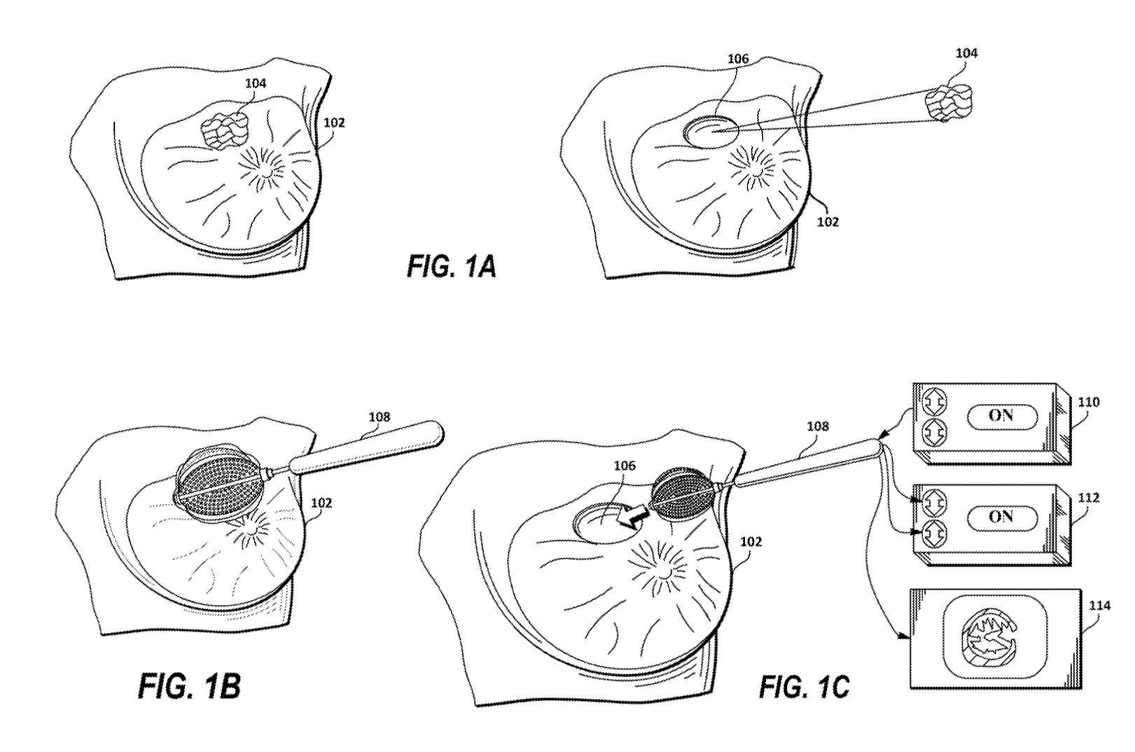

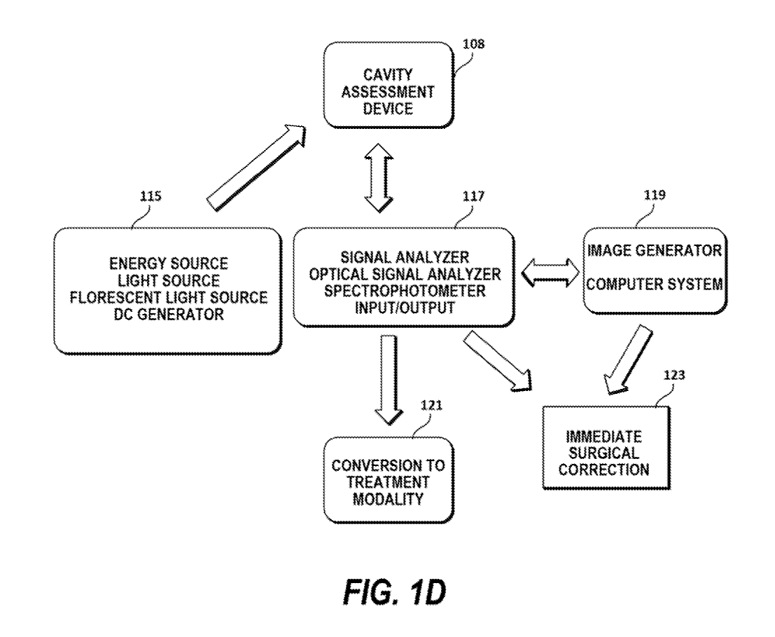

Turning now to the figures, FIG. 1A-1C are diagrams that show a surgical procedure and a general view of present cavity assessment device and system and some of the constituent elements thereof, according to embodiments of the present inventions. As shown in FIG. 1A, a breast (an example of tissue with which embodiments of the present invention may be practiced) is shown at 102 and a tumor within the breast is shown at reference numeral 104. During standard lumpectomy, a tumor 104 had been surgically resected from solid tissue that then forms a corresponding surgical resection cavity. Most commonly, the solid tissue is breast tissue that has been surgically treated to remove a tumor 104, leaving the resection cavity 106, as shown in FIG. 1B. The present assessment device 108 may be positioned through an opening created during the tissue resection surgery. In most cases, the surgery will be performed in the open manner so that the assessment device 108 may be easily positioned in the cavity 106 from which the tumor 104 has been removed. In case of the minimally invasive surgery, the present tissue characterization device or catheter 108 may be introduced through a small tissue channel formed during a minimally invasive procedure to remove the tumor 104. According to an embodiment of the present inventions, a method for characterizing tissue surrounding a surgical resection cavity 106 in solid tissue includes steps of introducing a tissue assessment device (also interchangeably called catheter herein) 108 into an interior of the cavity 106 as shown in FIG. 1C, through an open incision or through a narrow access path, and characterizing at least a portion of the surrounding tissue using the tissue assessment device 108—that is, at least the surface of the tissue that defines the post-surgical cavity 106. According to embodiments of the present invention, the characterization may include determining a presence and distribution of cancerous cells (or masses thereof) within the cavity 106. The characterization of the tissue of and within the cavity 106 may include, for example, viewing an image or a representation of a selected tissue characteristic on a display 114 coupled to the catheter 106. The selected characteristic that is displayed on the display 114 may include, for example, an image of the cavity illuminated by a visible light source within the distal portion (that portion of the catheter 108 that is inserted within the cavity 106) of the tissue assessment device 108. In this case, the light source (which may be a constituent part of the catheter 108, insertable therein or otherwise connectable thereto) may be coupled to a power supply or external light source 110. The selected characteristic may also be or include a visual representation of some electrical characteristic of the tissue within the cavity 106, such as, for example, conductivity or impedance. In this case, the catheter 108 may include electrodes coupled to a power supply or signal generator 112.

FIG. 1D is a block diagram of steps of the present tissue assessment method and other aspects thereof, according to embodiments of the present invention. As shown therein, the cavity assessment device 108 may be coupled to an energy source, light source, fluorescent light source (described hereunder) and/or a power or signal generator, collectively shown at reference number 115. Such sources generally provide the input required for the assessment modality contemplated. For example, the light source may be a visible light source or a source of infrared light, depending upon the assessment or treatment agent introduced within the cavity. The cavity assessment device or catheter 108 may also be coupled to a signal analyzer (electrical or optical), a spectrophotometer or other input/output device, collectively shown at 117. Such devices may be coupled to an image generator, a computer system and/or generally to any device or system enabling the surgeon to visualize the cavity and the changes elicited therein through the delivery of electrical signals, dyes, reagents and the like by the present cavity assessment device 108.

Depending upon the output of 117 and/or the images generated and displayed by the computer system, the present tissue assessment device may be used as a treatment modality (through administration, for example, of a therapeutic agent, or the inducement of tissue ablation through heat or cold or radiation), as shown at 121. In cases wherein the visualization of the cavity reveals, for example, large remaining cancerous regions within the cavity, immediate surgical correction may be indicated, as shown at 123.

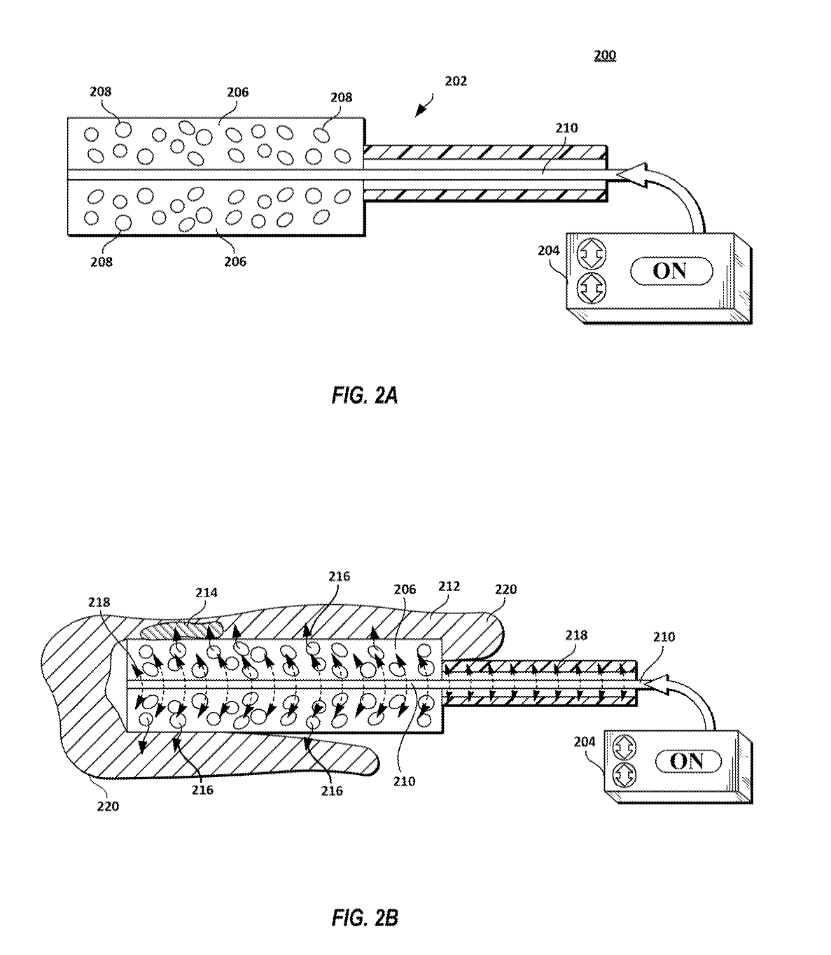

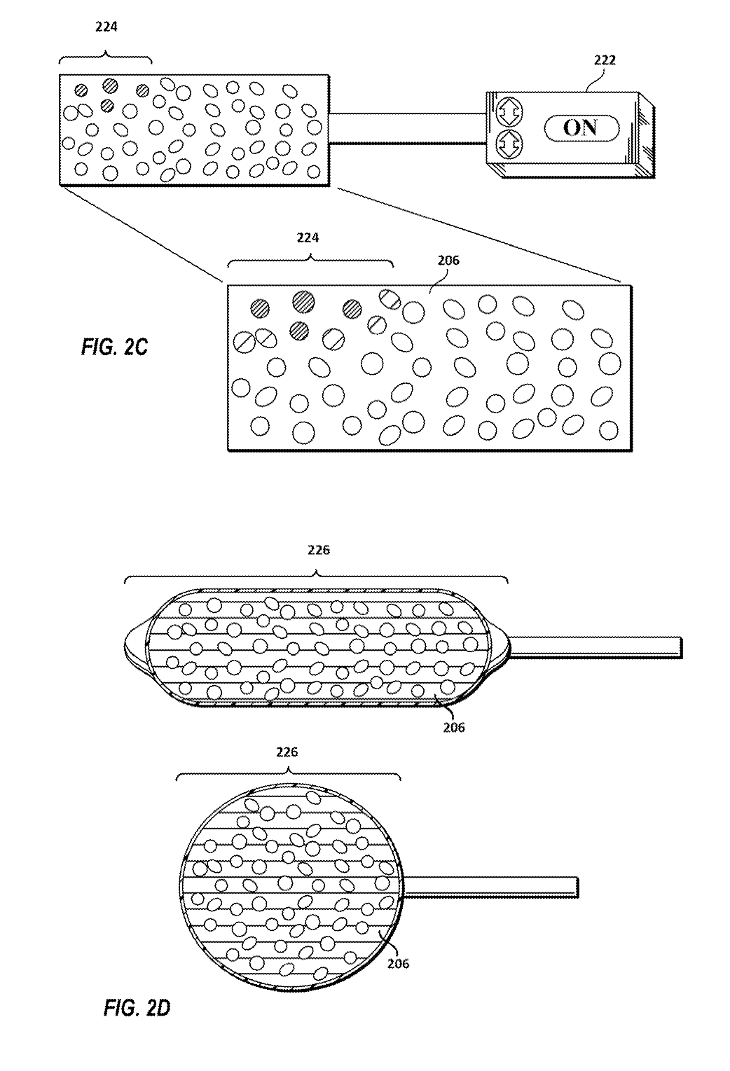

FIGS. 2A-2D illustrate tumor detection based on changes in analyzed tissue pH, according to embodiments of the present inventions. As shown in FIG. 1, an embodiment of the present inventions may be characterized as a bio-photometric cavity assessment system 200. The system 200 may include an assessment device or catheter 202 and a light source 204. The assessment device 202 may include a porous three-dimensional matrix 206 that includes immobilized pH sensitive dye 208. The pH sensitive dye 208 may be pre-loaded within the porous three dimensional matrix 206 or may be supplied thereto from a reservoir and supplied on demand. The light source 204 may supply a, for example, fiber optic light guide 210 that is positioned in a central lumen of the cavity assessment device 202, as shown in FIGS. 2A and 2B. The cavity assessment device 202 may also include a central expandable balloon having an outer surface on which is disposed the three-dimensional porous matrix 206. Such a balloon may be collapsed to facilitate positioning of the device 202 within the cavity and expanded to substantially fill the volume of the cavity.

FIG. 2B shows the cavity assessment system 202 inserted into a natural or manmade cavity or lumen within tissue 220. As shown, the tissue 220 may include residual cancerous, abnormal or pre-cancerous tissue 214. Such remaining cancerous tissue 214 may have been missed during a lumpectomy procedure, for example, in which less than perfect margins were achieved. After the device 202 is inserted within tissue 220, the immobilized pH sensitive dye 208 contained within and on the surface of the porous three-dimensional matrix 206 comes into contact with and is delivered to the interior surface of the cavity within the tissue 220, as indicated by the solid arrows 216. As cancerous tissues tend to have a different pH than surrounding healthy tissues, the delivered pH-sensitive dye 208 will react differently to the remaining cancerous tissue 214 than it will to the surrounding healthy tissue 220. Such difference may manifest itself as a difference in color, for example. The dye may also include a selected antibody having an affinity for cancerous tissues, which will cause a difference or gradient in the uptake of the dye in cancerous tissues. In any event, differences in the nature of the tissue under test may manifest themselves as differences in color and/or saturation, as the dye reacts to cancerous tissues differently than it does with healthy tissue 220. To visualize such differences, the light source 204 may be activated, which causes the centrally-located fiber optic channel 210 to illuminate the interior of the three-dimensional matrix 206. If the porous three-dimensional matrix (and the central expandable and collapsible balloon, if present) is made sufficiently transparent, the light from the energized fiber optic channel 210 will be transmitted therethrough, to illuminate the sidewalls of the cavity to which the pH-sensitive dye 208 has been delivered. In place of the fiber optic 210, a scope may be inserted within the central lumen of the assessment device 202, to enable a direct viewing of the light transmitted through the porous matrix 206 and reflected from within cavity on a display. Alternatively, and as shown in FIG. 2C, an optical signal analyzer 222 may be utilized, to detect malignant areas 24 having decreased pH levels using spectrophotometric signals.

Note that the characterization of the tissue (e.g., malignant or benign) may be carried out in situ, that is, within the cavity, while the cavity assessment device is inserted within the cavity. Alternatively, the characterization of the tissue may also be carried out ex vivo, that is, after the present cavity assessment device has been removed from the body. Indeed, a simple visual examination of the removed three-dimensional matrix 206 will reveal areas of different color and/or color saturation, which are indicative of cancerous tissues within the cavity at locations corresponding to their location on the three-dimensional matrix 206. The locations of the differences in color in the matrix 206 directly correlate with their corresponding locations of the cancerous tissues within the cavity. For example, the location of color and/or saturation changes suggested at 224 in FIG. 2C in the matrix 206 give the surgeon a strong indication that cancerous tissues may be present at the far distal end of the cavity. The orientation of the device within cavity and the location of changes in the color and saturation of the pH-sensitive dye may also be used to more precisely locate the areas of concern within the cavity. Therefore, the distal portion of the device 202 may become a physical three-dimensional map of the location of remaining cancerous tissues within the cavity.

FIG. 2D gives two additional examples of the many possible shapes of the distal portion 226 of the present cavity assessment device 202. The top drawing in FIG. 2D shows a distal portion that is shaped somewhat like a right-cylinder, and may be well suited to like shaped post-surgical cavities or natural or manmade lumens. The bottom drawing in FIG. 2D shows a roughly spherical distal portion 2D, suitable for cavities formed by cutting out a surface of revolution, for example. Of course, these shapes are merely exemplary and not limiting. The three-dimensional matrix 206 may be manufactured into most any shape that accommodates both specific procedures and specific excisional surgical instruments. Alternatively still, the surgeon may manually trim and shape the three-dimensional matrix 206 to best conform to a specifically-shaped cavity that he or she has just created.

As noted above, the distal portion 226 of the cavity assessment devices 202 may include an internal expandable and collapsible balloon, with the three-dimensional porous matrix 206 disposed on the outer surface thereof. The collapsible and expandable nature of such devices, coupled with the different shapes of the distal portions 226, allows the distal portions 226 to closely conform to the shape of the volume delimited by the surface of the post-surgical cavity. Indeed, the distal portion 226 may be expanded to not only closely conform to the surface of the cavity, but also to exert some measure of force thereon. Such physical pressure facilitates not only the delivery of the pH-sensitive dye, therapeutic agent or reagent from the porous matrix 206, but also increases the uptake of the pH-sensitive dye, therapeutic agent or reagent by the surrounding tissues. In turn, this yields better results, as more of the dye, therapeutic agent or reagent is delivered from the porous matrix and taken up by the tissues of the cavity sidewalls.

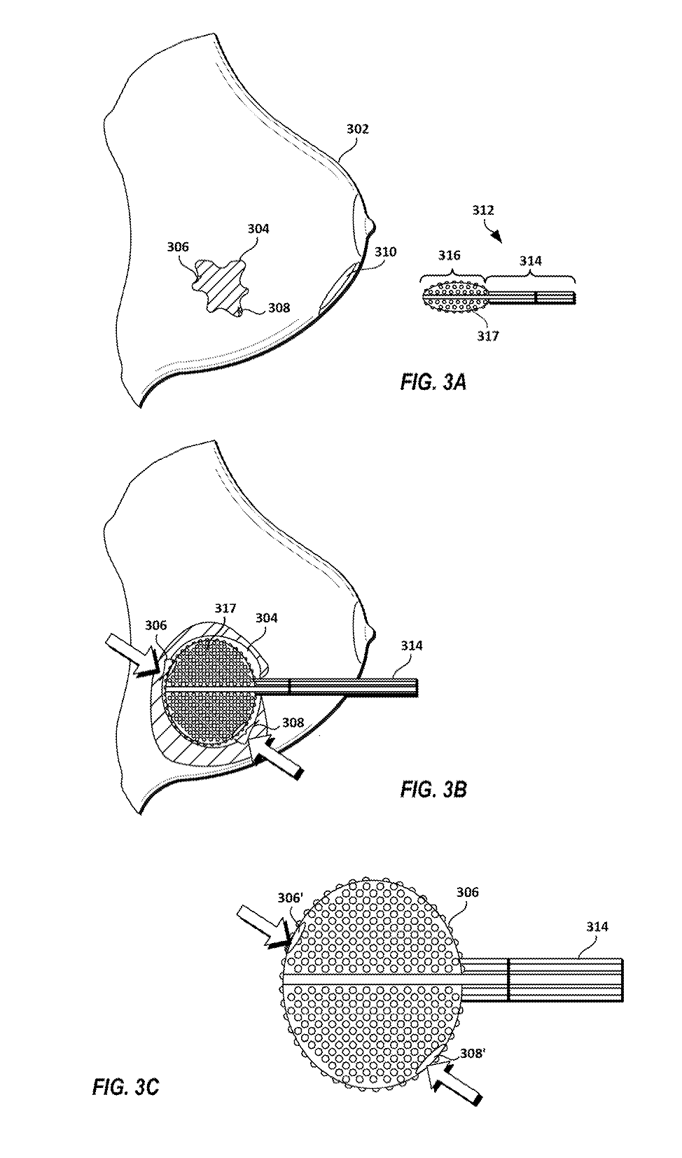

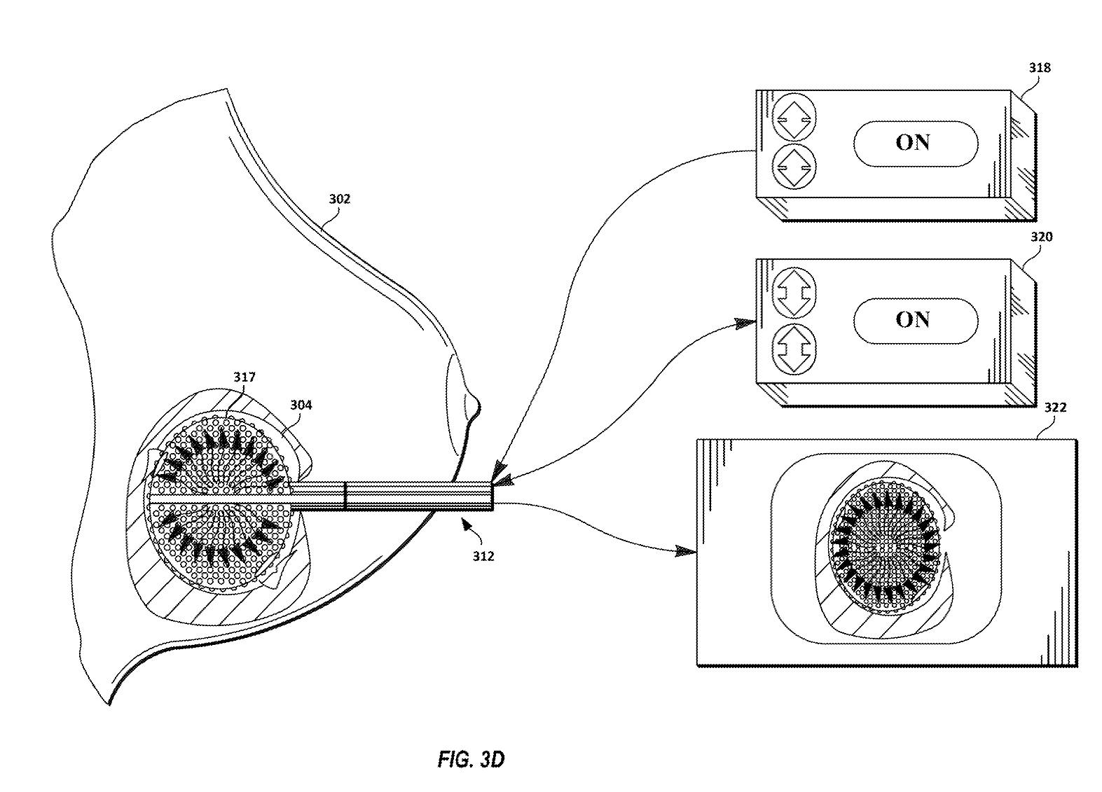

FIGS. 3A-3C illustrate a balloon-based cavity assessment device and a method for detecting remaining cells within a post-surgical cavity, based on changes in a reagent that is sensitive to abnormal cells (such as cancer cells, for example), according to embodiments of the present invention. Reference 302 represents a breast in which a post-surgical cavity 304 has been made through an opening 310. This embodiment calls for providing a catheter 312 having a proximal portion 314 and a distal portion 316. The distal portion 316 may include an expandable and collapsible balloon whose outer surface 317 may be provided with, coated or otherwise coupled with a reagent that is sensitive to abnormal cells. The expandable and collapsible balloon itself may be coated or coupled with the reagent or the balloon may support, on its outer surface, a porous matrix that is coupled or pre-loaded with (or otherwise configured to deliver) a reagent that is sensitive to abnormal cells. FIGS. 3A and 3B, for purposes of illustration, show that the cavity 304 includes two areas containing abnormal (e.g., cancerous) cells, shown at 306 and 308. The balloon or other enlargeable structure may be inserted within the cavity 304 in its collapsed state and then expanded in situ within the cavity 304 so that the balloon surface conforms closely to the surrounding tissue. Such close conformance is advantageous, both during the characterization of the tissue and to help create a symmetric, regular shape for the cavity which helps assure potential subsequent treatment, particularly from brachytherapy. The balloon may be contracted or collapsed to facilitate removal of tissue assessment device from the surgical resection cavity 304 at the end of the cavity assessment procedure and in case of a consequent treatment. Alternatively, the distal portion 316 may be detachable from the proximal section of the catheter 312, and the distal portion 316 used for immediate or subsequent treatment, such as to deliver therapeutic agents to the cavity site following the assessment of the cavity 304. The distal portion 316 may further be biodegradable.

Returning now to FIGS. 3A and 3B, the distal portion 316 of the catheter 312 is first inserted into the cavity 304 within the breast (or other biological tissue) with the distal portion 316 in the collapsed state shown in FIG. 3A. Once the distal portion 316 of the catheter 312 has been inserted within the cavity 304, the balloon of the distal portion 316 may be expanded so that the outer surface 317 thereof comes into intimate contact with a surface of the cavity, as shown in FIG. 3B. In this state, the reagent coupled to the balloon comes into contact (and may be pushed against) the surface(s) of the cavity 304. After waiting a (generally short) length of time sufficient for the reagent to react to the tissue with which it has been put in contact, the surface 317 of the distal portion 316 of the catheter 312 will have, in the illustrative and exemplary scenario developed in FIGS. 3A-3C, two localized and distinct regions 306′ and 308′ on the surface 317 of the distal portion 316 where the reagent has reacted to the presence of remaining abnormal cells 306 and 308 within the cavity 304.

The balloon of the distal portion 316 may now be deflated and the catheter 312 removed from the cavity 304. The distal portion 316 may now be re-expanded. In such a state, the expanded balloon now acts as a physical three-dimensional map to any remaining abnormal cells within the cavity 304. Indeed, the surface 317 of the balloon now includes regions 306′ and 308′ in which the reagent has reacted with remaining abnormal cells within the cavity 304 and changed appearance (e.g., color, saturation, appearance, etc.). If the orientation of the catheter within the cavity is noted and maintained when extracted from the cavity, the surgeon will benefit from a one-to-one correspondence between the indications 306′, 308′ of abnormal cells on the surface 317 of the balloon and the corresponding locations within the cavity 304 of the actual abnormal cells. Note that any system of orientation registration may be used to maintain correspondence of orientation when the device is inserted in and retracted from the cavity 304. For example, markings on the device 312 may be used for orientation registration both within the body and outside of the body.

FIG. 3C shows the indications 306′ and 308′ on the surface of the balloon, which correspond directly to the locations of the abnormal cells 306 and 308 within the cavity 304, as further emphasized by the arrows in both FIGS. 3B and 3C, which are located in corresponding locations in the cavity and on the surface of the balloon and are oriented in like manner.

Now that the surgeon has what amounts to an accurate three-dimensional map of remaining cells within the cavity 304, he or she may then proceed to treat the cavity using the reagent-coated surface of the expanded balloon as a map to corresponding locations of remaining abnormal cells within the cavity. Such treatment may include, for example, further resection of tissue within the cavity at locations indicated by a corresponding location on the surface of the balloon where the reagent reacted to the presence abnormal cells within the cavity. Such treatment may optionally be followed by administration of chemotherapy agents and/or the administration of radiation in situ. The reagent may be or may include, for example, a dye (such as a pH-sensitive dye, a photosensitive dye such as, for example, a fluorescent dye and/or a fluorescent dye and a selected antibody having an affinity to abnormal cells).

FIG. 3D illustrates a cavity assessment system utilizing balloon mapping technique based on visual changes in color for the respective areas of the balloon surface using computerized image analysis and reconstruction. As shown therein, the catheter 312 may be coupled to a visible light source 318, a fluorescent light source 320 (for use in illuminating fluorescent dyes) and/or other sources such as an infra-red light source, for example. As described earlier, the distal portion of the catheter may be at least partially transparent or translucent to the light emitted by the light source (e.g., 318 or 320). Moreover the light (in whatever form) may be transmitted through a central lumen to the distal portion of the catheter 312. Therefore, light emitted within the central lumen of the catheter will be at least partially transmitted through the at least partially transparent or translucent distal portion (for example through a partially transparent porous matrix to which a reagent has been coupled) and onto the surface of the cavity. If such is visualized on a display 322, the resulting image constitutes a map of the cavity 304, showing in particular regions where the reagent has reacted with abnormal cells and thus have a different appearance than those regions where the reagent has come into contact with only normal cells. The surgeon or operator may then utilize this information to switch to a treatment modality (using the same device 312) wherein, for example, chemotherapy or brachytherapy agents are administered (e.g., through the same or a different central lumen) to the cavity, optionally without removing the distal portion of the catheter from the cavity 304. The proximal portion of the catheter 312 may be detached from its distal portion if the chemotherapy or brachytherapy agents are to be left within the breast for an extended period of time. Alternatively, the practitioner may decide that surgical resection is indicted, whereupon he or she may freeze and store the image(s) on the display 322 and use it or them as a guide to the location where additional tissue should be resected.

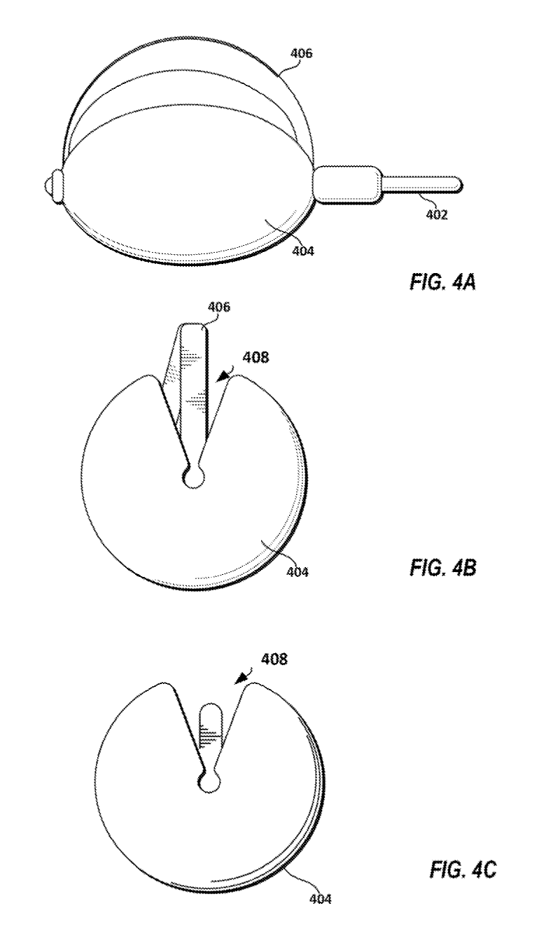

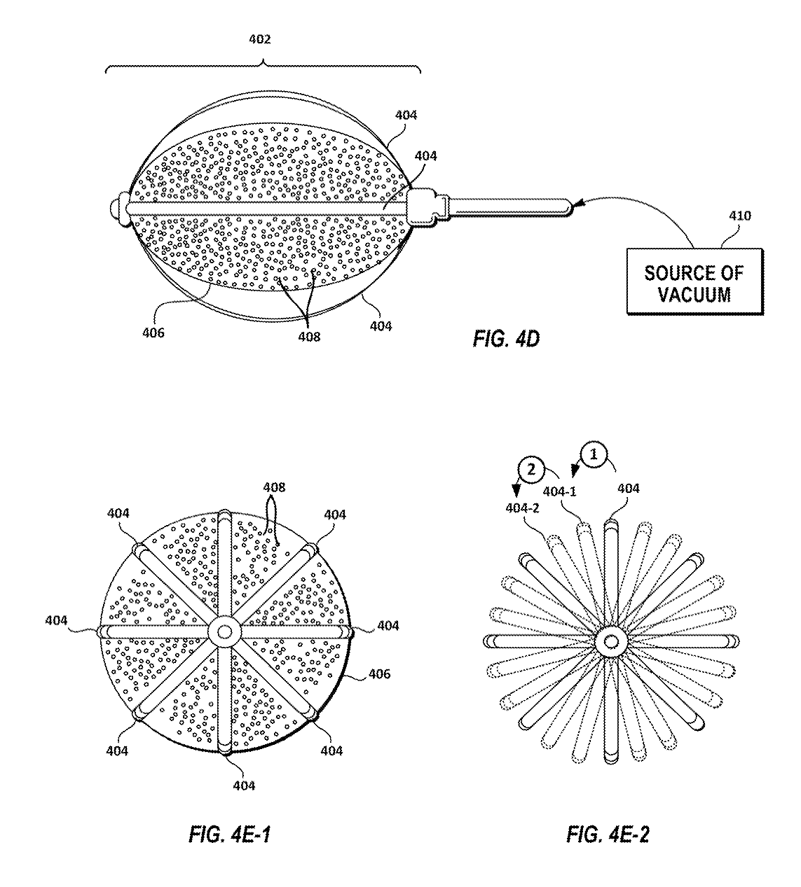

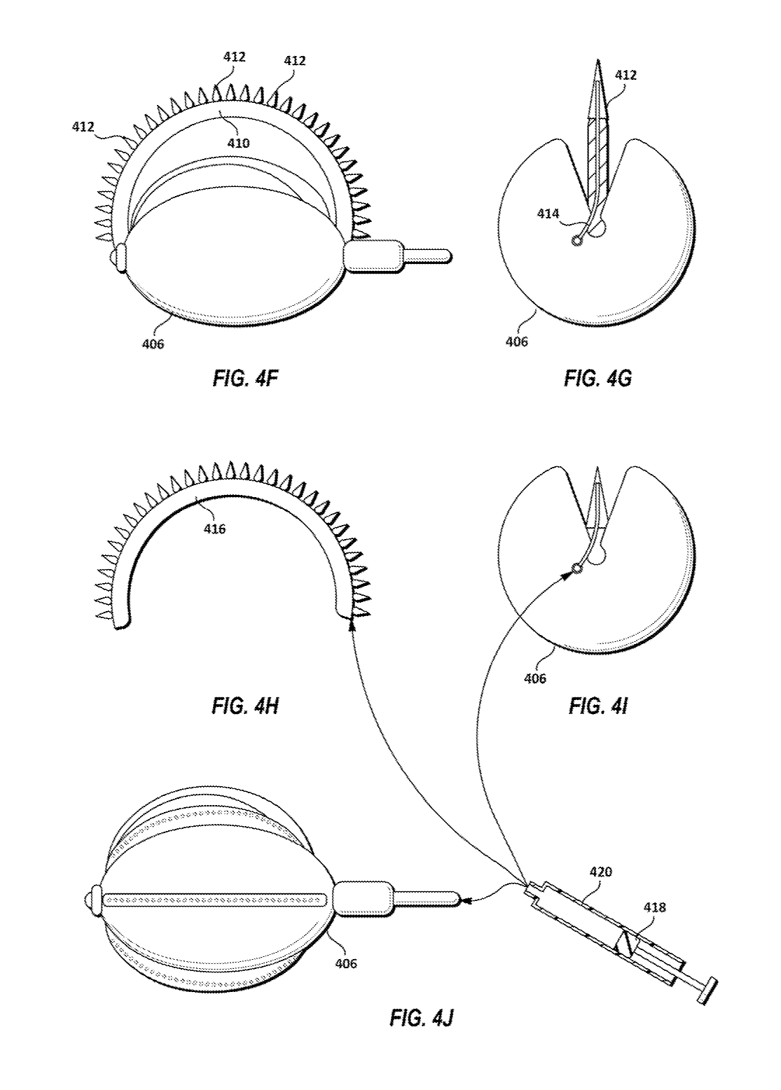

FIGS. 4A and 4B show a distal portion of a tissue assessment catheter, according to further embodiments of the present invention. The catheter may include a proximal portion 402 and a distal portion 404. The distal portion, in this embodiment, may include a cavity expander having a pre-set geometry (e.g., size and shape). When the cavity expander has a fixed geometry, its size may be about equal to or slightly greater than the volume of tissue resection cavity. The cavity expander may be substantially rigid or may be somewhat compliant. In this way, the fixed geometry structure will fit or slightly distend the cavity to help assure close conformance with the surface of the cavity into which it is to be inserted. The fixed diameter structure can be frangible, collapsible, or otherwise destructible so that it may be reduced in size prior to removing the tissue assessment device from the surgical resection cavity. The size and shape of such rigid cavity expander may be selected according to the size and shape of the cavity. As shown in FIG. 4B, the cavity expander of the distal portion 404 may include surfaces that define one or more radially oriented notches therein, such as best shown at reference numeral 408 in FIGS. 4B and 4C. The notch 408 may be configured to enable a carrier or loop 406 to retract therein or expand therefrom. During insertion (or rotation) of the distal portion 404 within the cavity, the loop 406 may be caused to assume its retracted state shown in FIG. 3C, so as not to impede the smooth insertion of the device within the cavity or rotation therein. Once in place within the cavity, the loop 406 may be caused to assume, via a suitable actuator coupled to the proximal portion 402 of the catheter, its expanded state, shown in FIG. 4B.

The loop 406 may be coated with a reagent that has an affinity for abnormal cells such as cancer cells. The loop 406 alternatively may support a layer having a three-dimensional matrix to which a suitable reagent has been coupled. By expanding the loop 406 and causing the loop to come into intimate contact with the tissue within the cavity, the reagent and tissue may be brought into contact with one another across a band that is co-extensive with the area of contact of the carrier 406 with the tissue at the surface of the cavity. By retracting the carrier 406 within the notch 408 as shown in FIG. 3C and rotating at least the distal portion of the catheter, the notch 408 and carrier 406 will face another region of the cavity. The loop 406 may then be caused to again assume its expanded state and create another band of tissue within the cavity that has been exposed to the reagent coupled to the loop 406. This process may be repeated to generate a plurality of bands of tissue having been placed into intimate contact with the reagent on the carrier 406. Collectively, these bands provide the surgeon with indications of the locations of abnormal cells within the cavity. Indeed, the cavity may then be illuminated and either directly visualized or imaged on a computer display, with the areas of the surface of the cavity having reacted to the reagent having a different appearance than those areas of the surface of the cavity that did not react to the reagent.

It is to be noted that the present embodiments are not limited to a single notch 408 and a single carrier 406. Indeed, the rigid cavity expander of the distal portion 404 may include a plurality of circumferentially spaced and radially oriented notches and a corresponding plurality of carriers 406 collapsible therein and expandable therefrom.

FIGS. 4D and 4E-1 show an alternative embodiment of the distal portion of the cavity assessment device according to embodiments of the present inventions. FIG. 4 shows a side view of the cavity assessment device (the proximal portion thereof not being shown in any detail) and FIG. 4E-1 is a front facing view of the distal portion of the present cavity assessment device. As shown, the distal portion 402 may include a plurality of carriers/loops 404 that are retractable in close conformity with the tissue expander portion 406 as shown at FIG. 4E-1 or expandable away from the tissue expander portion 406, as shown in FIG. 4D. Suitable actuator(s) (not shown) for the loops 404 may be disposed on the proximal portion of the catheter. The tissue expander 406 may be rigid and may have a preset geometry. The tissue expander 406 may also include surfaces that define a plurality of vacuum orifices 408 that are in fluid communication with a source of vacuum 410. Indeed, a vacuum may be drawn in the interstitial region between the surface of the tissue expander portion 406 and the tissue to draw the tissue against an outer surface of the tissue expander 406. Applying a vacuum to draw the tissue against the tissue expander structure further promotes close conformance between the tissue and the tissue expander structure, allowing for enhanced imaging and treatment. The vacuum drawn through these vacuum orifices 408 tends to facilitate the delivery of reagent from the carriers 404 as they are expanded (as shown in FIG. 4D) and pressed onto the facing tissue within the cavity. As noted above, the carriers (which need not be configured as loops) may be coated with a suitable reagent or may support a layer of a three-dimensional matrix to which a reagent is coupled and/or delivered.

In use, the loops 404 come into contact with a plurality of bands of tissue within the cavity and apply corresponding bands of reagent to the tissue. The majority of the surface of the cavity may be applied with the reagent by sequentially expanding the carriers 404, retracting the carriers 404, rotating at least the distal portion 402 of the catheter, re-expanding the carriers 404 and repeating the process. Indeed, as shown in FIG. 4E-2, as few as two such iterations may be sufficient to obtain a substantially complete reagent-derived picture of the surface of the cavity. Indeed, in FIG. 4E-2, the initial position of the carriers 404 is shown at 404 in solid lines. After a first rotation (of about 15 degrees), the position of the carriers (in dashed lines) is shown at 404-1 and after a second rotation (of about 15 degrees), the position of the carriers 404 is shown at 404-2. As can be seen, even as few as two rotations and subsequent application of reagent to the surfaces of the cavity facing the respective carriers 404 is effective to cover most of the interior of the cavity and thus provide the surgeon with a highly detailed map of any remaining abnormal tissues within the cavity.

FIGS. 4F-4H show other embodiments of the distal portion of the cavity assessment device according to the present inventions. FIG. 4F shows a cavity expander 406 and a single loop 410 equipped with a plurality of needle-like structures 412. As shown in FIG. 4G in which a single needle-like structure is shown in cross-section, each of the needle-like structures 412 may include a central lumen through which a conduit 414 may be disposed. The conduits 414 may be coupled to a source of abnormal cell sensitive agent (or reagent, as the terms are used interchangeably herein). Alternatively, the loop(s) 410 may be fitted with a flexible sleeve 416 that is configured to contain a volume of reagent 418. In any event, whether the needle-like structures are fitted with a conduit 414 or are in direct fluid communication with a volume of reagent as shown in FIG. 4H, the needle-like structures disposed on or formed integrally with the loop(s) 410, are configured to deliver an amount of reagent 418 to facing tissue within the cavity when the carrier(s) 410 are expanded from their retracted state (FIG. 4I) to their expanded state (FIG. 4G). As shown in FIG. 4J, a syringe 420 or functional equivalent, loaded with a suitable reagent 418, may be coupled to the catheter to deliver the reagent 418 to the interior surfaces of the post-surgical cavity through the needle-like structures of the carrier(s) 410.

Alternatively, the needle-like structures 412 may be coupled to a conductive wire or trace within conduit 414, for applications based on an electro-physical response, such as is the case wherein conductivity or tissue impedance is being measured and used as an indicator or possible remaining cancerous tissues within the cavity. In such an application, the plurality of needle-like structures act as electrodes, delivering electrical energy to tissues within the post-surgical cavity.

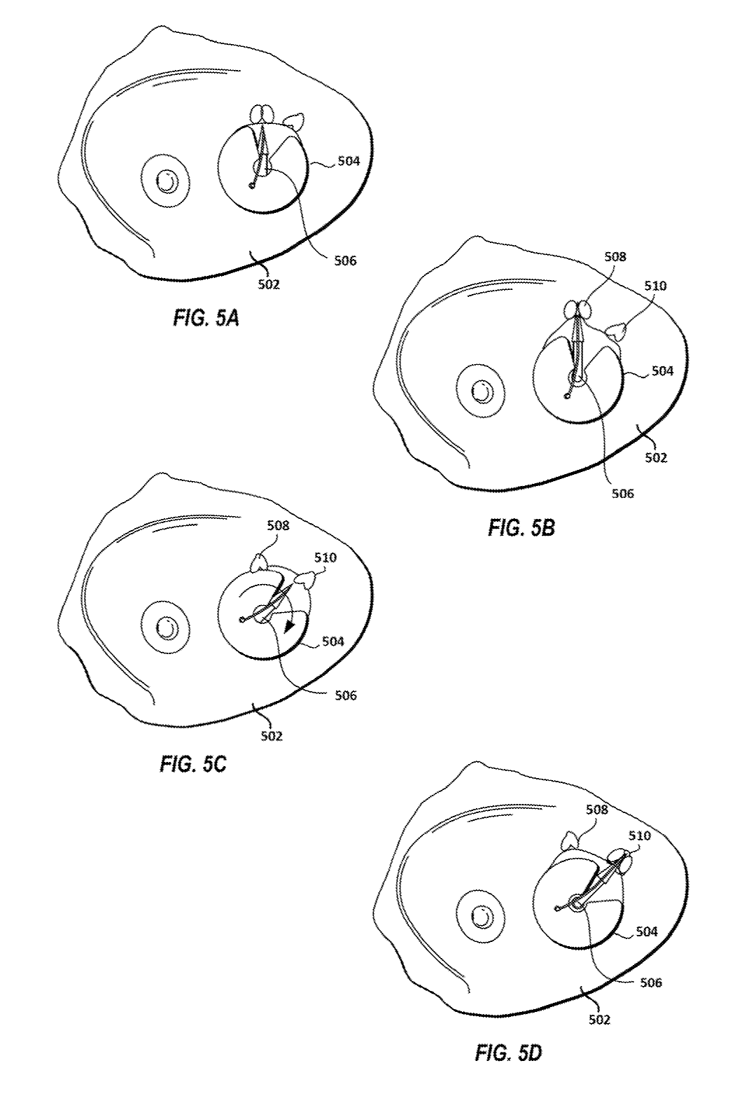

FIGS. 5A-5D are cross-sectional representations of the distal portion of a cavity assessment device according to an embodiment of the present invention, in use. As shown, FIG. 5A is a cross-sectional view of the distal portion 504 inserted within breast tissue 502 with the carrier(s) in its or their retracted state, such that the needle-like structures 506 thereof do not protrude from the tissue expander 504. As shown in FIG. 5B, the carrier is actuated to its expanded configuration, in which the plurality of needle-like structures comes into contact with the facing tissue within the cavity. In this illustrative view, the carrier or one of the carriers has been expanded so that needle-like structures contact or are inserted into a mass of abnormal cells 508 remaining after a lumpectomy. During the cavity assessment phase, the needle-like structures may be caused to inject reagent (abnormal tissue sensitive agent, marking agent, dye, pigment, etc.) into this mass 508, as shown in FIG. 5B. Thereafter, the carrier(s) may be retracted, so that the plurality of needle-like structures 506 are no longer in contact with or inserted in the cavity walls, as shown in FIG. 5C, and at least the distal portion of the assessment device is rotated, as also shown in FIG. 5C. Thereafter, as shown in FIG. 5D, the carrier(s) may again be caused to expand, to cause the needle-like structures 506 to again come into contact with and/or penetrate the facing tissue within the cavity to again inject reagent (this time, in tissue mass 510). This procedure of expand, inject, retract, rotate, expand and inject may be repeated as many times as necessary to generate a useful mapping of the tissue characteristics of the surface of the cavity. Providing the distal portion with a plurality of loops or carriers (as the terms are used interchangeably herein), each equipped with a linear array of needle-like elements will substantially decrease the number of iterations necessary to develop a useful mapping of the interior of the cavity. During a subsequent treatment phase, the surgeon may utilize the positional information gathered from the above-detailed steps to map any remaining abnormal cells within the cavity and surgically resect further tissue from the cavity, or introduce a therapeutic agent (e.g., a chemotherapy drug or a source of radiation) into the cavity, through a central lumen thereof and/or through the linear array of needle-like structures, without removing the distal portion of the catheter from the cavity.

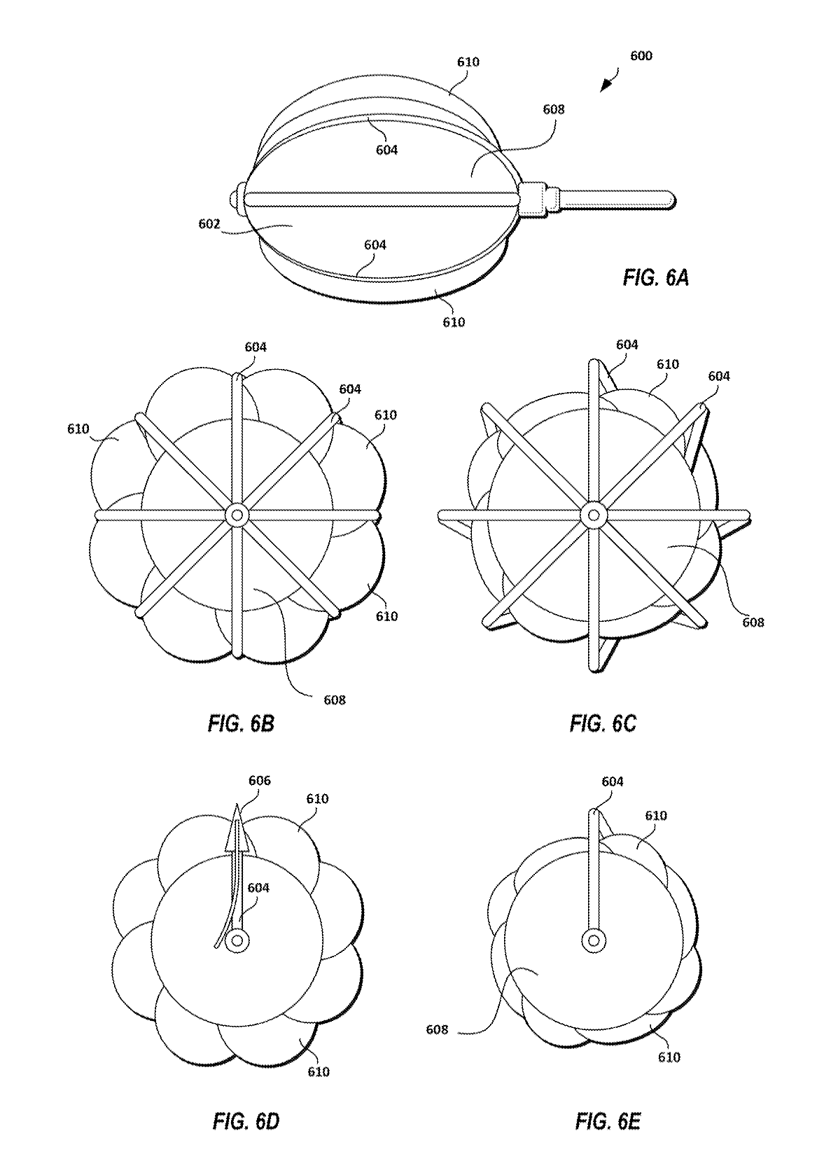

FIGS. 6A-6C illustrate yet another embodiment of a cavity assessment device, according to a further embodiment of the present inventions. As shown in FIG. 6A, the distal portion of the catheter 600 may include a tissue expander 608, which may be selectively expandable and collapsible and/or may have a pre-set geometry (e.g., preset size and shape). A plurality of inflatable and collapsible (e.g., generally cylindrical-shaped) balloons 610 may be provided circumferentially around the tissue expander 608. A carrier 604 may be provided between adjacent two collapsible and expandable balloons. As shown in FIG. 6B, when the balloons 610 are inflated, the carriers 604 are nestled between adjacent ones of the balloons and do not protrude from the highest radial extent thereof. However, as shown in FIG. 6C, when the balloons are caused to assume their deflated or collapsed state, the carriers or loops 604 then become the highest radially extending structures. In this state, the carriers may be caused to contact the facing surface of the cavity and cause some reagent to be applied thereto. In this case, the cavity expander 608 may be provided with surfaces defining vacuum orifices that are coupled to a source of vacuum, as shown, for example, in FIG. 4D. The application of vacuum within the cavity causes the surface of the cavity to be drawn and adhere somewhat to the tissue expander 608, thereby assuring good contact between the carrier 604 and the walls of the cavity. According to an embodiment of the present inventions, the catheter includes a combination of a tissue expander 608 with a preset geometry and collapsible and expandable balloons 610. This combination enables the distal portion of the catheter to assume one of two baseline profiles: pre-set geometry tissue expander with expanded balloons and pre-set geometry tissue expander with collapsed balloons. These profiles aid in insertion of the distal portion of the catheter into the cavity (tissue expander 608 and collapsed balloons 610) and in subsequent assessment and/or treatment (tissue expander 608 and expanded balloons 610).

Yet another embodiment of the present invention is a variation on the structure detailed relative to FIG. 6, but in which the carriers 604 are configured to selectively assume a collapsed configuration as shown in FIG. 6B against the expanded balloons 610 and to assume an expanded configuration in which the carriers 604 expand above the expanded balloons to contact the facing tissue within the cavity. Other embodiments are possible.

Indeed, during insertion of the catheter 600 into the cavity, the balloons 610 may be caused to assume their expanded state, to ensure that the carriers 604 do not come into intimate contact with the access path or the cavity. Once inside the cavity, the balloons 610 may be caused to selectively assume their respective collapsed state, to thereby expose the carriers 604 (coated or otherwise provided with suitable reagent, marking fluid or pigment) to the surface of the cavity, advantageously together with the application of vacuum. If rotation of at least the distal portion of the catheter 600 is desired, the balloons 610 may be caused to assume their respective expanded state. As shown at FIGS. 6D and 6E, embodiments with fewer carriers (such as a single carrier 604 as shown in FIG. 6E) is possible, as are embodiments that include one or more needle like structures 606 (as shown in FIG. 6D). As foreshadowed above, the tissue expander 608 may include a plurality of surfaces that define vacuum orifices that are configured to couple to a source of vacuum, as was shown in FIGS. 4D and 4E-1. Therefore, where the tissue expander 608 is not covered by one of the plurality of balloons 610, the vacuum orifices may be effective in drawing the surface of the cavity toward the carriers 604, to further facilitate the delivery of dye (pH-sensitive, photo-sensitive (such as fluorescent dye, for example)) and/or other reagents to the surface oldie cavity.

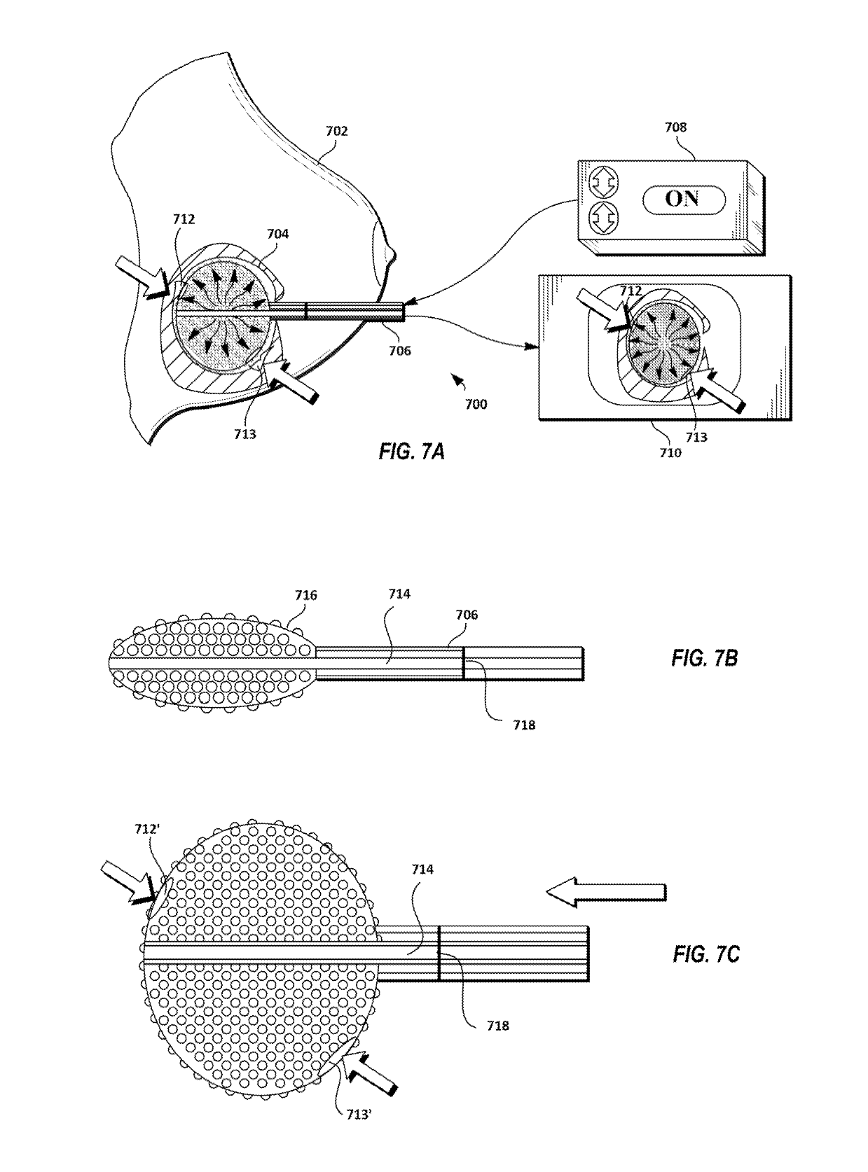

FIG. 7A illustrates a cavity assessment system 700 utilizing selective fluorescent or photodynamic agent uptake and subsequent assessment based on visual changes in color for the respective areas of the cavity inner surface using direct visualization and/or computerized image analysis and reconstruction, according to an embodiment of the present invention. As shown in FIG. 7B, the distal portion of the catheter 706 may include a selectively expandable and collapsible balloon 716 that is provided with, coupled to or otherwise configured to deliver fluorescent dye and/or fluorescent dye and a selected antibody having affinity for cancerous cells. For example, the surface of the balloon 716 may support a three-dimensional matrix to which a dye/reagent/pigment/therapeutic agent is coupled. In the specific embodiment of FIG. 7B, the dye may be initially contained with a reservoir 714 and pushed to the balloon 716 via a plunger 718. In the configuration illustrated in FIG. 7B, the balloon 716 is in its collapse state and the plunger 718 in a proximal position. This configuration is suitable for insertion of the catheter into tissue.

As shown in FIGS. 7A and 7C, the catheter 706 is in a configuration wherein it has been expanded in vivo, within the cavity 704(FIG. 7A) and in a configuration wherein it has been extracted from the cavity 704 and re-expanded (FIG. 7C). As shown in FIG. 7A, the distal portion of the catheter 706 has been inserted within the cavity 704 and the plunger 718 pushed to a more distal position. Pushing the plunger 718 in the distal direction reduces the available volume in the reservoir 714, forcing the dye to the surface of the balloon 716 and out into the cavity 704. After a predetermined (generally short) period of time during which the fluorescent dye is up-taken by any abnormal cells within the cavity 704, a source of fluorescent light 708 is activated, shining fluorescent light into the cavity through the at least partially transparent or translucent balloon 716 and/or three-dimensional porous matrix thereon, as shown in FIG. 7A. Those locations within the cavity 704 that include abnormal cells will react differently than areas having only normal cells and this difference may be visualized directly, or through image processing techniques, as suggested by 710. The display at 710 may, therefore, display a representation of the light reflected within the cavity 704, and indicate the presence, if any, of abnormal cells through their different appearance relative to other areas of the cavity (e.g., a color gradient indicative of locations of abnormal cells). In this case, the cavity includes abnormal cells 712 and 713 and such abnormal cells show up on the representation shown by the display 710. Likewise, when the catheter 700 is removed from the body and the balloon 716 thereof re-expanded as shown in FIG. 7C, it can be readily seen that the fluorescent dye (optionally with a selected antibody having an affinity for cancerous cells) reacted with the cancerous cells upon application of the fluorescent light from source 708, at the locations 712′ and 713′ indicated by the arrows. Such an expanded balloon bearing the indications 712′ and 713′ of activated reagents (such as fluorescent dyes) is now quite literally a physical, three-dimensional map to the interior of the cavity 704 where the features of interest are the locations any remaining cancerous cells within the cavity. Such locations may be read directly from the expanded distal portion of the catheter, provided that the orientation of the catheter within the body is maintained or otherwise taken into account when the catheter is removed from the cavity.

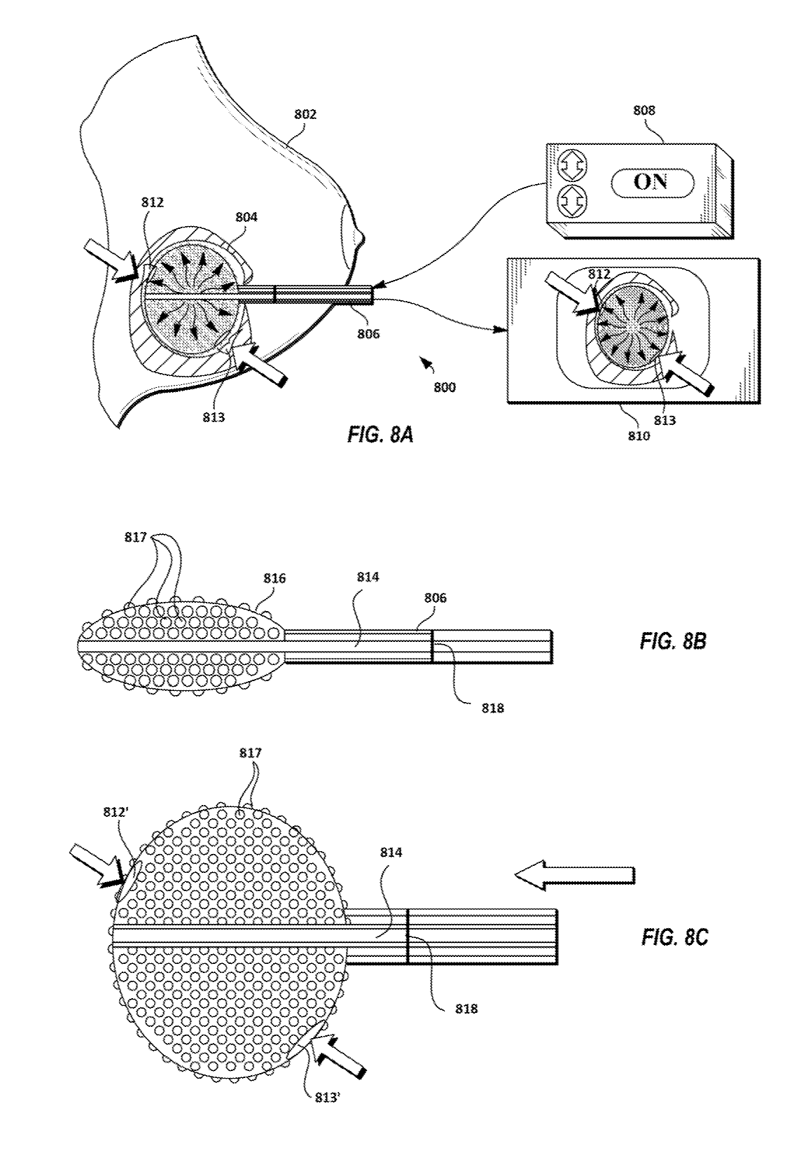

In a specific embodiment, the tissue assessment device may include one or more electro-conductive elements, where the elements are coupled with expandable aspects of the present device, a source of an iontophoretic dye and are configured to enable dye to be directed or injected into tissue within the cavity. Based on a difference in impendence between normal and cancerous cells, iontophoretic dye distribution may be measured in the tissue lining the interior of the cavity, optionally producing a three-dimensional image of the cavity tissue on a display. In this embodiment, shown in FIG. 8, the distal portion of the catheter, which may include an expandable and collapsible balloon 816, includes or is otherwise provided with a plurality of electro-conductive actuators 817 on a surface of the balloon 816. The electro-conductive actuators are coupled to electrical source 808. As shown in FIGS. 8A and 8C, the catheter 806 is in a configuration wherein it has been expanded in vivo within the cavity 804 (FIG. 8A) and in a configuration wherein it has been extracted from the cavity 804 and re-expanded (FIG. 8C). As shown in FIG. 8A, the distal portion of the catheter 806 has been inserted within the cavity 804 and the plunger 818 pushed to a distal position. Pushing the plunger 818 in the distal direction reduces the available volume in the reservoir 814, thereby forcing the iontophoretic agent contained therein to the surface of the balloon 816 and out into the cavity 804, in contact with the now (or soon to be) energized electro-conductive actuators disposed on the surface of the balloon 816. After a predetermined short period of time during which the iontophoretic agent is up-taken by any abnormal cells within the cavity 804, light may be provided within a central lumen in the catheter 806, thereby illuminating the cavity 804 through the at least partially transparent or translucent balloon 816 and/or three-dimensional porous matrix thereon, as shown in FIG. 8A. Those locations within the cavity 804 that include abnormal cells will react differently to the activated iontophoretic dye than areas having only normal cells and this difference may be visualized directly, or through computerized image processing techniques, as suggested by 810. The display at 810 may, therefore, display a representation of the light reflected within the cavity 804, and indicate the presence of abnormal cells (if any remain in the cavity 804) through their different appearance relative to other areas of the cavity (e.g., a color gradient indicative of locations of abnormal cells). In this case, the cavity 804 includes abnormal masses of cells 812 and 813 and such abnormal masses of cells show up on the representation shown by the display 810. Likewise, when the catheter is removed from the body and the balloon 816 thereof re-expanded as shown in FIG. 8C, it can be readily seen that the iontophoretic dye reacted to the application of the electrical energy to the electro-conductive actuators 817 at the locations referenced by 812′ and 813′, as indicated by the arrows. Such expanded balloon bearing the indications 812′ and 813′ of activated reagents (such as fluorescent dyes) is and may then be used as a physical, three-dimensional map to the interior of the cavity 804, provided that the orientation of the catheter within the body is maintained or otherwise taken into account.

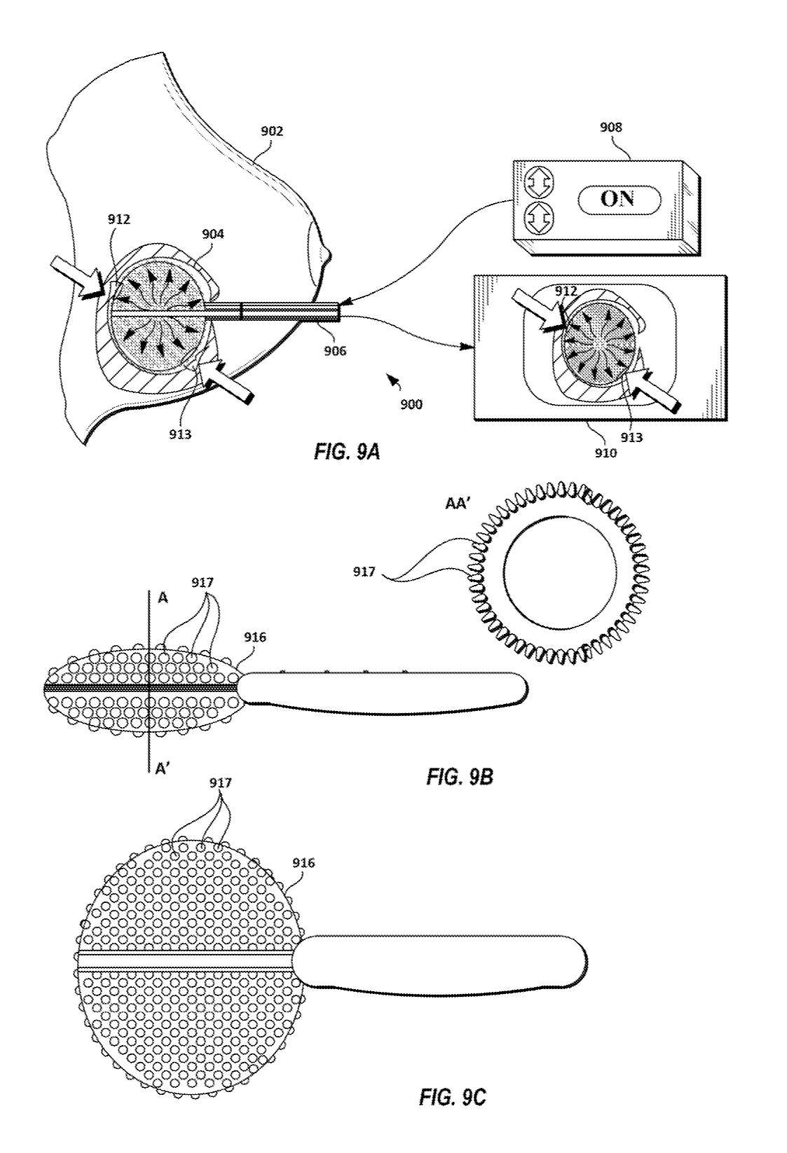

Yet another embodiment the tissue assessment device includes an inflatable balloon or an expandable structure equipped with a plurality of metallic members connected to an external power generator. Upon expansion of the device, the metallic members make contact with the interior tissue lining of the cavity. A small current or diagnostic energy is selectively applied to the metallic members to provide a localized impedance or conductivity mapping of the cavity walls. It is generally believed that the cancerous cells can be differentiated from their sounding healthy tissue by having varying levels of the impedance. This variation may be detected by the metallic members of the tissue assessment device and transmitted to an external equipment to determine the exact location of cancerous cells within the cavity. When positional accuracy of the tissue assessment device is confirmed and it is verified that the metallic members are opposed against the cancerous cells, then a higher amount of current or therapeutic energy may be applied to the device during in a treatment phase, for a predetermined period of time sufficient to cause obliteration of the cancerous cells and result in a subsequent treatment. FIG. 9A illustrates a cavity assessment system utilizing selected electrophysiological properties such impedance or conductivity, based on a differential in conductivity between benign and malignant tissues that can, in turn, be converted into visual changes in color (for example) for the respective areas of cavity inner surface using computerized image analysis and reconstruction. In this embodiment, the distal portion of the catheter, which may include an expandable and collapsible balloon 916, includes or is otherwise provided with a plurality of electro-conductive elements 917 on a surface of the balloon 916. According to one embodiment, the plurality of electro-conductive elements 917 may be or include conductive needle-like structures that act as electrodes on the surface of the balloon 916, as best depicted in the cross-sectional view along AA′ in FIG. 9B. According to other embodiments, the plurality of electro-conductive elements 917 may be or include conductive raised pads. The electro-conductive elements 917 are coupled to electrical source 908. As shown in FIGS. 9A and 9C, the catheter 906 is in a configuration wherein it has been expanded in vivo, within the cavity 904 (FIG. 9A) and in a configuration wherein it has been extracted from the cavity 904 and re-expanded (FIG. 9C). As shown in FIG. 9A, the distal portion of the catheter 906 has been inserted within the cavity 904. Thereafter, electrical energy may be supplied to all or selected ones of the elements 917 and the conductivity and/or impedance of the tissue in contact with the energized electrodes measured and recorded in a computer system 910. As cancerous tissues exhibit comparatively lesser conductivity and greater impedance than healthy tissue, different colors (for example) may be assigned to those areas of the cavity 904 exhibiting such decreases in conductivity and increases in impedance than are assigned to tissues exhibiting a baseline conductivity and impedance. This information may then be converted into visual changes in color for the respective areas of cavity inner surface using computerized image analysis and reconstruction. Such visual images may then be displayed on a display of the computer system, as shown at 910 and acted upon.

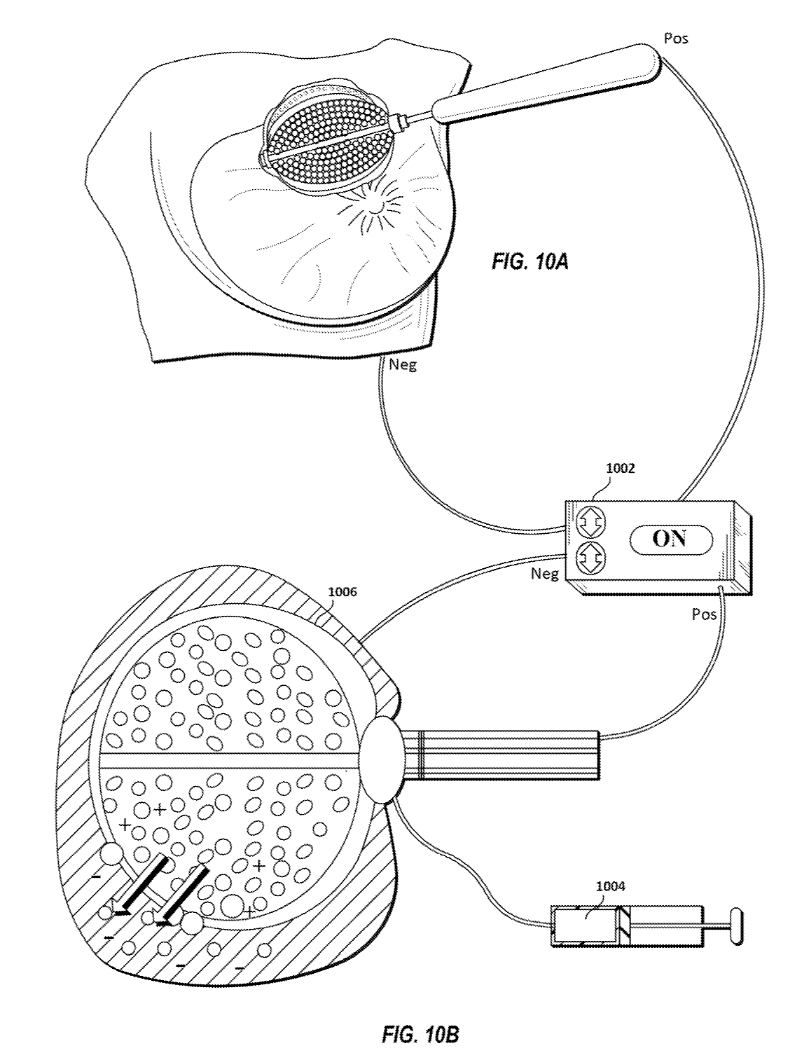

According to a further embodiment of the present inventions, an electric field may be set up between the cavity assessment device and the surrounding tissue, to drive a charged iontophoretic agent into the cavity walls. As shown in FIGS. 10A and 10B, a DC generator may have one polarity coupled to the catheter and to the iontophoretic agent (such as an iontophoretic dye) contained therein and another polarity coupled to the surrounding tissue. The iontophoretic agent, under the influence of the electric field, will be driven into the tissue lining the surface of the post-surgical cavity. In turn, localized masses of cancerous cells within and on the surface of the cavity 1006 will react to the iontophoretic dye and will be dyed a different color than surrounding healthy tissue and such colors may be visualized, mapped and used for subsequent treatment decisions.

According to still other embodiments, the distal portion of the catheter may be formulated in situ using biodegradable materials. Such biodegradable distal portion may be removably attached to the proximal portion acting as an introducer shaft, which would not be biodegradable, thus allowing removal of the proximal portion after the cavity assessment is complete. Such biodegradable distal portion may be loaded with a therapeutic agent (e.g., source of radiation or chemotherapy). The distal portion may then be left in place, providing sustained release of the therapeutic agent and gradual degradation in situ over a preselected period of time, typically weeks or months.

As shown and described herein, the tissue of the post-surgical cavity may be characterized in a variety of ways, including differential impendence mapping, fluorescent histochemical staining, pH measurements, or a variety of other techniques. For example, the tissue may be characterized by optical imaging combined with fluorescent tumor specific dye delivery to the cavity interior lining. By delivery of the tumor specific fluorescent agent from the exterior of the distal aspect of the present tissue assessment device to the inner lining tissue of the cavity, accumulation of the agent will occur in the cancerous cells. Absorbed fluorescent dye may be detected using light delivered via optical fiber or from a miniature scope inserted into the cavity to indicate that some tumor cells may have been left behind in the original surgical resection. In such instances, it is advantageously possible to immediately access the cavity and surgically remove additional tissue, before the patient has left the operating room.

According to further embodiments, different assessment modalities (such as optical and impedance mapping, for example) may be employed and the results thereof graphically superimposed upon one another prior to application of therapeutic current to make certain that the tissue assessment device and its metallic members are accurately positioned against the cancerous cells.

In particularly advantageous embodiments of the present inventions, three-dimensional images (or two-dimensional projections of three-dimensional images) may be obtained, which allow for improved detection of residual tumor cells. In addition to detecting residual disease, creation of the three-dimensional images will allow for more accurate guidance for the immediate or subsequent treatment.

According to still further embodiments, in addition to characterizing the tissue in the resection cavity, the methods of the present inventions may further include marking at least a portion of the tissue surrounding the resection cavity with a pigment. Such marking may include delivering a pigment or a dye through injection to the selected residual cancer cells which may remain via, for example, a plurality of the needle-like structures described herein. Marking the tissue surrounding the surgical resection cavity may include positioning a marking device into the cavity based on the feedback obtained from the cavity assessment device. The marking device can be combined or coupled with the present assessment device.

In addition to characterizing the tissue in the resection cavity, the methods of the present invention may further include treating at least a portion of the tissue surrounding the resection cavity. Such treatment may include delivering local radiation therapy or local chemotherapy to the tissue, particularly to treat residual cancer cells that may remain in the cavity. Treating the tissue surrounding the surgical resection cavity may be carried out immediately subsequent to tissue assessment and characterization, using the same device as was used for tissue assessment. Indeed, the local radiation therapy or local chemotherapy may be delivered to the cavity through the distal portion of the present tissue assessment devices, in the same manner as the reagents are delivered to the cavity during the assessment phase. For brachytherapy, radiation sources may be delivered to the cavity via the or one of the central lumens in the tissue assessment device, without removing the device from the cavity between the assessment and treatment steps, thereby combining an assessment modality with a treatment modality in a single device.

According to still further embodiments, once tissue assessment is completed using any of the methods described herein above, a therapeutic device may be introduced to treat any detected remaining cancerous cells. Such a therapeutic device may include an inflatable balloon with electrically or optically transmissive segments covering its surface. Once inside the previously characterized tissue cavity, the balloon may be expanded to make intimate contact with interior walls of the cavity. The previously obtained tissue assessment information that may include an optical mapping of cancerous cells is provided at this time to ensure exact positioning of the conductive segments of the balloon against the cancerous cells. Therapeutic energy may then applied to the conductive or transmissive exterior segments of the balloon using an external generator such as radio frequency, microwave or laser. The power level and duration of exposure may be optimized to cause necrosis of the cancerous cells.

While the foregoing detailed description has described preferred embodiments of the present invention, it is to be understood that the above description is illustrative only and not limiting of the disclosed invention. Those of skill in this art will recognize other alternative embodiments and all such embodiments are deemed to fall within the scope of the present invention. Thus, the present invention should be limited only by the claims as set forth below.

What is claimed is:

1. A method of characterizing tissue, comprising:
providing a catheter defining a proximal portion and a distal portion, the distal portion including a carrier that is configured to deliver an agent that is sensitive to abnormal cells;
inserting the catheter in a cavity within the tissue;
causing the carrier to bring the abnormal cell sensitive agent into contact with a surface of the cavity, and
illuminating the surface of the cavity.

2. The method of claim 1, further comprising:
rotating the distal portion of the catheter within the cavity and causing the carrier to bring the abnormal cell sensitive agent into contact with another region of the surface of the cavity prior to the illuminating step.

3. The method of claim 1, further comprising:
characterizing the tissue of the interior surface of the cavity depending upon a characteristic of a light reflected from within the cavity or absorbed by the surface of the cavity.

4. The method of claim 3, wherein the characterizing step includes determining whether the surface of the cavity includes cancer or other abnormal cells depending upon characteristics of the light reflected from within or absorbed by the cavity.

5. The method of claim 1, further comprising:
bringing the distal portion of the catheter into close conformance with a surface of the cavity using a source of vacuum.

6. The method of claim 1, wherein the providing step is carried out with the abnormal cell sensitive agent including a pH-sensitive dye.

7. The method of claim 1, wherein the providing step is carried out with the abnormal cell sensitive agent including a fluorescent dye.

8. The method of claim 1, wherein the providing step is carried out with the abnormal cell sensitive agent including a fluorescent dye and a selected antibody having an affinity to abnormal cells.

9. The method of claim 1, wherein the providing step is carried out with the catheter including a plurality of carriers that include pH-sensitive elements.

10. The method of claim 1, wherein the providing step is carried out with the distal portion of the catheter including a light source.

11. The method of claim 1, wherein the illuminating step is carried out by inserting a scope within the catheter to the cavity, the scope including a light source.

12. The method of claim 10, further including:
displaying an image produced from light reflected from within or absorbed by the cavity on a display coupled to the scope.

13. The method of claim 6, further comprising:
determining a gradient of pH across the surface of the cavity, the gradient being indicative of a presence and distribution of cancerous or other abnormal cells within the cavity.

14. The method of claim 1, wherein the providing step is carried out with the carrier including a plurality of needle-like structures configured to deliver the abnormal cell sensitive agent to the cavity.

15. The method of claim 14, wherein the providing step is carried out with the distal portion including a lumen in fluid communication with a reservoir of abnormal cell sensitive agent and the plurality of needle-like structures.

16. The method of claim 1, wherein the providing step is carried out with the carrier including a balloon that is configured to deliver the abnormal cell sensitive agent to a surface of the cavity.

17. The method of claim 16, wherein the balloon is configured to be selectively expandable and collapsible.

18. The method of claim 16, wherein the balloon is configured to deliver the abnormal cell sensitive agent over a surface of the cavity that is substantially coextensive with an external surface of the balloon.

19. The method of claim 16, wherein the providing step is carried out with a surface of the balloon including surfaces that define a plurality of openings, the plurality of openings being coupled to a source of vacuum.

20. The method of claim 1, wherein the providing step is carried out with the distal portion of the catheter including a cavity expander having a preset geometry, the cavity expander including surfaces that define a plurality of openings that are configured to couple to a source of vacuum.

21. The method of claim 20, further comprising a plurality of selectively expandable and collapsible balloons.

22. The method of claim 20, wherein a size and shape of cavity expander is configured to substantially match a size and shape of the cavity, but for an opening configured to enable the carrier to selectively collapse therein and emerge therefrom to bring the abnormal cell sensitive agent into contact with the surface of the cavity.

23. A catheter, comprising:
a proximal portion and a distal portion;
a carrier, the carrier being configured to deliver an agent that is sensitive to abnormal cells to a surface of a cavity within biological tissue;
a source of illumination configured to illuminate abnormal cell sensitive agent delivered to the surface of the cavity, and
an interface configured to couple to a display, the interface being configured to enable the display to display an image representative to light reflected from or absorbed by the abnormal cell sensitive agent delivered to the surface of the cavity.

24. The catheter of claim 23, further comprising:
a plurality of carriers configured to deliver abnormal cell sensitive agent to a surface of the cavity.

25. The catheter of claim 23, wherein the source of illumination is coupled to the distal portion of the catheter.

26. The catheter of claim 23, wherein at least the distal portion includes a surface configured to define an interior lumen through which a scope including a light source is insertable.

27. The catheter of claim 23, wherein the carrier includes a plurality of needle-like structures configured to deliver the abnormal cell sensitive agent to at least the surface of the cavity.

28. The catheter of claim 27, wherein at least the distal portion includes a surface that defines a lumen in fluid communication with a reservoir of abnormal cell sensitive agent and the plurality of needle-like structures.

29. The catheter of claim 23, wherein the carrier is coupled with an abnormal cell sensitive agent.

30. The catheter of claim 29, wherein the abnormal cell sensitive agent includes a pH-sensitive dye.

31. The catheter of claim 29, wherein the abnormal cell sensitive agent includes a fluorescent dye.

32. The catheter of claim 29, wherein the abnormal cell sensitive agent includes a fluorescent dye and a selected antibody having an affinity to abnormal cells.

33. The catheter of claim 23, further including:
a balloon, wherein the carrier includes an outer surface of the balloon, and wherein at least a portion of the outer surface of the balloon is configured to deliver the abnormal cell sensitive agent to a surface of the cavity.

34. The catheter of claim 33, wherein the balloon is configured to be selectively expandable and collapsible.

35. The catheter of claim 33, wherein the balloon includes surfaces that define a plurality of openings, the plurality of openings being configured to couple to a source of vacuum.

36. The catheter of claim 23, further comprising:
a cavity expander having a preset geometry.

37. The catheter of claim 36, further including a plurality of selectively expandable and collapsible balloons disposed on the cavity expander.

38. The catheter of claim 36, wherein a size and shape of cavity expander is configured to substantially match a size and shape of the cavity, but for an opening configured to enable the carrier to expand and emerge therefrom to bring the abnormal cell sensitive agent into contact with a surface of the cavity facing the carrier.

39. The catheter of claim 38, wherein the cavity expander defines a plurality of radially oriented openings, each configured to enable a respective carrier to expand and emerge therefrom and come into contact with a surface of the cavity.

40. The catheter of claim 23, wherein the carrier includes a porous matrix that is coupled with the abnormal cell sensitive agent.

41. The catheter of claim 23, wherein the carrier includes an expandable and collapsible loop and wherein the proximal portion of the catheter includes an actuator coupled to the carrier.

42. The catheter of claim 23, further comprising:
a plurality of balloons coupled to the distal portion, and
a plurality of carriers, wherein the plurality of balloons are each configured to assume an expanded state in which each of the plurality of carriers is nestled between at least two of the expanded balloons and a collapsed state in which the plurality of carriers are configured to come into contact with a surface of the cavity.

43. A method of characterizing tissue, comprising the steps of:
providing a catheter defining a proximal portion and a distal portion, the distal portion including a surface that is configured to contact a surface of a cavity within the tissue, the distal portion being configured to selectively elicit a physiological response from tissue within the cavity;
inserting the catheter in the cavity within the tissue;
causing the physiological response to occur, and
observing the physiological response and characterizing the tissue depending upon the observed physiological response.

44. The method of claim 43, wherein the providing step is carried out with the distal portion being configured to substantially conform to a shape and size of the cavity.

45. The method of claim 43, wherein the providing step is carried out with at least a portion of the distal portion being expandable and collapsible.

46. The method of claim 43, wherein the providing step is carried out with at least a portion of the distal portion having surfaces that define a plurality of vacuum orifices that are configured to couple to a source of vacuum, and wherein the distal portion is configured to drawn the surface of the cavity toward the distal portion when vacuum is applied.

47. The method of claim 43, wherein the providing step is carried out with the distal portion including a porous matrix.

48. The method of claim 43, wherein the wherein the providing step is carried out with the distal portion being configured to selectively elicit the physiological response using a pH-sensitive dye.

49. The method of claim 43, wherein the wherein the providing step is carried out with the distal portion being configured to selectively elicit the physiological response using a fluorescent dye.

50. The method of claim 43, wherein the wherein the providing step is carried out with the distal portion being configured to selectively elicit the physiological response using a fluorescent dye with a selected antibody having an affinity to abnormal cells.

51. The method of claim 43, wherein the wherein the providing step is carried out with the distal portion being configured to deliver a reagent that is sensitive to malignant cells.

52. The method of claim 43, wherein the wherein the providing step is carried out with the distal portion being configured to deliver a reagent having sensitivity to an ionic strength of the tissue within the cavity.

53. The method of claim 43, wherein the wherein the providing step is carried out with the distal portion being configured to deliver a reagent having sensitivity to conductivity of the tissue within the cavity.

54. The method of claim 43, wherein the wherein the providing step is carried out with the distal portion being configured to deliver a reagent configured to cause light to reflect or be absorbed differently from or by the tissue within the cavity depending upon a characteristic of the tissue.

55. The method of claim 43, wherein the surface of the distal portion includes at least one needle-like structure configured to deliver at least one of a reagent and a therapeutic agent to tissue within the cavity.

56. The method of claim 43, wherein the surface of the distal portion includes at least two electrodes and wherein the physiological response is electrical in nature.

57. The method of claim 56, wherein the physiological response is one of conductivity and impedance.

58. The method of claim 43, wherein the wherein the providing step is carried out with the distal portion being configured to deliver an iontophoretic agent to the cavity.

59. A method of mapping a post-surgical cavity, comprising:
providing a catheter, the catheter having a distal portion configured to substantially occupy a volume of space delimited by a surface of the cavity, the distal portion being at least partially translucent to a predetermined source of light, the distal portion being further configured to deliver a reagent from a surface of the distal portion to the surface of the cavity;
inserting the catheter within the cavity and delivering the reagent to at least a surface of the cavity, and
illuminating the cavity with the predetermined source of light through the at least partially translucent distal portion.

60. The method of claim 59, further comprising:
displaying an image of a light reflected from within the cavity on a display that is external and coupled to the catheter.

61. The method of claim 59, wherein the providing step is carried out with the distal portion including a porous matrix.

62. The method of claim 59, wherein the providing step is carried out with the distal portion in fluid communication with a reservoir of the reagent.

63. The method of claim 59, wherein the providing step is carried out with the distal portion being expandable such that it substantially occupies the volume of space delimited by the cavity and collapsible such that it then occupies a smaller volume than the volume delimited by the cavity.

64. The method of claim 59, wherein the providing step is carried out with the distal portion including surfaces that define vacuum orifices that are configured to couple to a source of vacuum, and wherein the inserting step includes a step of adhering the surface of the cavity to the distal portion using vacuum.

65. The method of claim 59, wherein the delivering step is configured to deliver the reagent to most of the entire surface of the cavity at the same time.

66. The method of claim 59, wherein the delivering step delivers the reagent across at least one band on a surface of the cavity, and wherein the delivering step further includes at least one step of rotating the distal portion within the cavity and re-delivering the reagent across at least one other band on a surface of the cavity.

67. The method of claim 59, wherein the providing step is carried out with the distal portion including a user-actuated collapsible and expandable loop, and with the distal portion defining a preset geometry that substantially conforms to a size and shape of the cavity, the distal portion further defining at least one radial notch within which the loop is configured to collapse and expand.

68. A catheter, comprising:
a proximal portion, and
a distal portion coupled to the proximal portion and configured to be inserted within a cavity within biological tissue, the distal portion including a portion that is at least partially transparent to a predetermined light and that is configured to deliver a reagent to the cavity, the distal portion being further configured to one of include and receive a source of the predetermined light to illuminate a surface of the cavity to which the reagent has been delivered through the at least partially transparent portion.

69. The catheter of claim 68, further comprising:
an interface, the interface being configured to couple to a display to enable light reflected from within the cavity to be represented on the display.

70. The catheter of claim 68, wherein the at least partially transparent portion includes a three dimensional porous matrix.

71. The catheter of claim 68, wherein the at least partially transparent portion is preloaded with the reagent.

72. The catheter of claim 68, wherein the at least partially transparent portion is in fluid communication with a reservoir of the reagent.

73. The catheter of claim 68, wherein the distal portion is expandable such that it substantially occupies the volume of space delimited by the cavity and is collapsible such that it then occupies a smaller volume than the volume delimited by the cavity.

74. The catheter of claim 68, wherein the distal portion includes surfaces that define vacuum orifices that are configured to couple to a source of vacuum.

75. The catheter of claim 68, wherein the at least partially transparent portion is configured to deliver the reagent to most of the entire surface of the cavity at the same time.

76. The catheter of claim 68, further comprising a first electrode coupled, to the distal portion that is configured to be electrically charged to a first polarity and a second electrode that is configured to couple to the tissue and that is configured to be charged to a second polarity, wherein the reagent is driven into the tissue when the first and second electrodes are charged and establish a potential difference therebetween.

77. A method, comprising:
providing a catheter having a proximal and a distal portion, the distal portion including an expandable and collapsible balloon whose outer surface is coupled to a reagent that is sensitive to abnormal cells;
inserting the distal portion of the catheter in a cavity within biological tissue with the distal portion in a collapsed state;
expanding the balloon so that the outer surface of the balloon comes into intimate contact with a surface of the cavity;
collapsing the balloon and retracting the catheter from the cavity;
expanding the balloon and treating the cavity using the reagent-coated surface of the expanded balloon as a map to corresponding locations of remaining abnormal cells within the cavity.

78. The method of claim 77, wherein the providing step is carried out with the reagent including a pH-sensitive dye.

79. The method of claim 77, wherein the providing step is carried out with the reagent including a fluorescent dye.

80. The method of claim 77, wherein the providing step is carried out with the reagent including a fluorescent dye and a selected antibody having an affinity to abnormal cells.

81. The method of claim 77, wherein the treating step includes resecting further tissue within the cavity at locations indicated by a corresponding location on the surface of the balloon where the reagent reacted to a presence abnormal cells within the cavity.

82. The method of claim 77, wherein the treating step includes delivering a therapeutic agent to the cavity.

83. The method of claim 82, wherein the therapeutic agent includes at least one of a source of radiation and a chemotherapy agent.

84. The method of claim 82, wherein the providing step is carried out with the distal portion including a surface that defines a central lumen and includes a plurality of needle-like structures on a surface of the balloon and wherein the treating step includes re-inserting the distal portion of the catheter in the cavity and delivering the therapeutic agent to the cavity through at least one of the central lumen and at least one of the plurality of needle-like structures.
